(19) United States
(12) Patent Application Publication
(10) Pub. No.: US 2015/0265821 A1
(43) Pub. Date: Sep. 24, 2015

(54) ASSEMBLIES AND METHODS FOR FLUID DELIVERY

(71) Applicant: SMITH & NEPHEW, INC., Memphis, TN (US)

(72) Inventor: Lei Shi, Mansfield, TX (US)

(21) Appl. No.: 14/438,312

(22) PCT Filed: Nov. 5, 2013
(86) PCT No.: PCT/US2013/068566

§ 371 Date: Apr. 24, 2015

Related U.S. Application Data
(60) Provisional application No. 61/722,492, filed on Nov. 5, 2012.

(51) Int. Cl. A61M 35/00; A61M 11/00

(52) U.S. Cl. CPC A61M 35/00; A61M 11/00; A61M 2202/07; A61M 2202/09; A61M 2205/10

(57) ABSTRACT

Assemblies and methods for fluid delivery (e.g., solutions, solutions comprising suspensions of cells, fibrinogen, thrombin, and the like), such as those, for example, configured to deliver a fluid to a body of a person. The assemblies comprise a first inlet channel (42); a second inlet channel (46); a first outlet channel (54); a second outlet channel (58); a first pump (66, 78); a second pump (66, 74); and a body (14) configured to be coupled to a first container and a second container such that an interior of the first container is in fluid communication with the first inlet channel, and such that an interior of the second container is in fluid communication with the second inlet channel; where if a first container with a fluid and a second container with a fluid, are coupled to the body, the assembly is configured upon at least one actuation to successively pump fluid from the first container, through the first inlet channel, and out the first outlet channel; and fluid from the second container, through the second inlet channel, and out the second outlet channel.

### CROSS-REFERENCE TO RELATED APPLICATIONS

This application claims priority to U.S. Provisional Patent Application 61/722,492 filed on Nov. 5, 2012, which is incorporated by reference in its entirety.

### BACKGROUND OF THE INVENTION

1. Field of the Invention

This invention generally relates to fluid delivery (e.g., solutions, solutions comprising suspensions of cells, fibrinogen, thrombin, and the like), and more particularly, but not by way of limitation, to assemblies and methods configured to deliver a fluid to a body of a person.

2. Description of the Related Art

Examples of fluid delivery apparatuses, assemblies, and methods are disclosed, for example, in U.S. Pat. Nos. 4,006,841; 5,509,575; 5,759,171; 5,980,866; 6,461,325; 6,835,186 and in U.S. Patent Publication No. 2012/0043347.

### SUMMARY OF THE INVENTION

This disclosure includes embodiments of fluid delivery assemblies and methods. For example, embodiments of the present fluid delivery assemblies and methods are configured to deliver a fluid (e.g., solutions, solutions comprising suspensions of cells, fibrinogen, thrombin, and the like) to a body of a person.

Some embodiments of the present fluid delivery assemblies comprise a plurality of inlet channels (e.g., a first inlet channel, a second inlet channel, a third inlet channel, or more), a plurality of outlet channels (e.g., a first outlet channel, a second outlet channel, a third outlet channel, or more), at least one valve (e.g., one, two, three, or more valves) coupled to the plurality of inlet channels and the plurality of outlet channels, the at least one valve configured to be actuated (e.g., by a button) between a plurality of configurations, each configuration permitting fluid communication between one of the plurality of inlet channels and one of the plurality of outlet channels and preventing fluid communication between the other(s) of the plurality of inlet channels and the other(s) of the plurality of outlet channels, a body coupled to the at least one valve and configured to be coupled to a plurality of containers (e.g., one, two, three, or more containers) such that an interior of each of the plurality of containers is in fluid communication with an inlet channel of the plurality of inlet channels, and at least one pump (e.g., one, two, or more pumps) coupled to the body such that if a plurality of containers with fluid are coupled to the body, the at least one pump is configured to pump from each of the plurality of containers, through an inlet channel of the plurality of inlet channels, and out of an outlet channel of the plurality of outlet channels.

Other embodiments of the present fluid delivery assemblies comprise a plurality of inlet channels (e.g., a first inlet channel, a second inlet channel, a third inlet channel, or more), a plurality of outlet channels (e.g., a first outlet channel, a second outlet channel, a third outlet channel, or more), a plurality of pumps (e.g., one, two, or more pumps), and a body configured to be coupled to a plurality of containers (e.g., one, two, or more containers) such that an interior of the plurality of containers is in fluid communication with an inlet channel of the plurality of inlet channels, where if a plurality of containers each having a fluid, are coupled to the body, the assembly is configured upon at least one actuation to successively pump fluid from each of the plurality of containers, through an inlet channel of the plurality of inlet channels, and out an outlet channel of the plurality of outlet channels.

In some embodiments, the at least one valve can alternate between the plurality of configurations with successive actuations of the assembly; and in other embodiments, the at least one valve can alternate between the plurality of configurations with one actuation of the assembly. In other embodiments, fluid can be pumped from each of the plurality of containers with successive actuations of the assembly; and in still other embodiments, the fluid can be pumped from each of the plurality of containers with one actuation of the assembly. In some embodiments, the plurality of outlet channels are adjacent, are parallel, and/or extend from the body of the assembly. In some embodiments, the plurality of outlet channels are configured to atomize a fluid (e.g., a fluid in the plurality of containers). The plurality of containers (e.g., each of the plurality of containers) are configured to contain a solution; and in some embodiments, the solution comprises a suspension of cells. For example, the solution can comprise fibrinogen and/or thrombin, but is not required to. The solution can also be delivered to a body of a person.

Some embodiments of the present methods comprise coupling a plurality of containers (e.g., one, two, or more containers) containing fluid to a fluid delivery assembly, where the fluid delivery assembly comprises a plurality of inlet channels (e.g., a first inlet channel, a second inlet channel, third inlet channel, or more), a plurality of outlet channels (e.g., a first outlet channel, a second outlet channel, a third outlet channel, or more), at least one valve (e.g., one, two, or more valves), where the at least one valve is configured to alternate between a plurality of configurations, each configuration permitting fluid communication between one of the plurality of inlet channels and one of the plurality of outlet channels and preventing fluid communication between the other(s) of the plurality of inlet channels and the other(s) of the plurality of outlet channels, and at least one pump (e.g., one, two, or more pumps); and actuating the at least one pump to separately pump from each of the plurality of containers, through a corresponding inlet channel, and out of an outlet channel.

Other embodiments of the present methods comprise coupling a plurality of containers (e.g., one, two, or more containers) containing fluid to a fluid delivery assembly, where the fluid delivery assembly comprises a plurality of inlet channels (e.g., a first inlet channel, a second inlet channel, a third inlet channel, or more), a plurality of outlet channels (e.g., a first outlet channel, a second outlet channel, a third outlet channel, or more), and a plurality of pumps (e.g., one, two, or more pumps); and actuating the plurality of pumps to separately pump from each of the plurality of containers, through a corresponding inlet channel, and out of an outlet channel.

Any embodiment of any of the assemblies and methods can consist of or consist essentially of—rather than comprise/include/contain/have—any of the described elements, features, and/or steps. Thus, in any of the claims, the term “consisting of” or “consisting essentially of” can be substituted for any of the open-ended linking verbs recited above, in order to change the scope of a given claim from what it would otherwise be using the open-ended linking verb. For purposes of “consisting essentially of,” in one non-limiting aspect, a basic and novel characteristic of the fluid delivery assemblies and methods disclosed in this specification includes the ability to spray a solution comprising cells to a person's body in such a manner that cells remain viable while also spraying a secondary composition to the body simultaneously or sequentially with the solution comprising cells.

The feature or features of one embodiment may be applied to other embodiments, even though not described or illustrated, unless expressly prohibited by this disclosure or the nature of the embodiments.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure or identical embodiments. Rather, the same reference number may be used to indicate a similar feature or a feature with similar functionality, as may non-identical reference numbers. The figures illustrate the described elements using graphical symbols that will be understood by those of ordinary skill in the art. The embodiments of the present fluid delivery assemblies and their components shown in the figures are drawn to scale for at least the embodiments shown.

FIG. 1 depicts a perspective view of one embodiment of the present fluid delivery assemblies.

FIG. 2 depicts a side view of another embodiment of the present fluid delivery assemblies.

FIG. 3 depicts a front view of the fluid delivery assembly of FIG. 2.

FIG. 4 depicts a side view of another embodiment of the present fluid delivery assemblies.

FIG. 5 depicts a top view of a valve of the fluid delivery assembly of FIG. 4.

FIG. 6 depicts a perspective view of another embodiment of the present fluid delivery assemblies.

FIG. 7 depicts a front view of the fluid delivery assembly of FIG. 6.

FIG. 8A-C depict a side view of the fluid delivery assembly of FIG. 6 having a plurality of outlet channels that are adjustable.

FIG. 9 depicts a side view of the fluid delivery assembly of FIG. 6 having a plurality of outlet channels that are non-adjustable.

FIG. 10 depicts a side view of the fluid delivery assembly of FIG. 6, where the assembly comprises a cone coupled to the assembly around the plurality of outlet channels.

FIG. 11-13 depict side views of embodiments of the present fluid delivery assemblies having various button configurations.

FIG. 14 depicts a perspective view of another embodiment of the present fluid delivery assemblies.

FIG. 15 depicts a front view of the fluid delivery assembly of FIG. 14.

FIG. 16 depicts a side view of the fluid delivery assembly of FIG. 14.

FIG. 17 depicts a user pressing a button of an embodiment of the present fluid delivery assemblies.

FIG. 18 depicts a user pressing a button of another embodiment of the present fluid delivery assemblies.

FIGS. 19A-19E depict another embodiment of the present fluid delivery assemblies comprising a linear cam drive.

FIGS. 20A-20I depict another embodiment of the present fluid delivery assemblies comprising a rack drive.

FIGS. 21A-21B depict another embodiment of the present fluid delivery assemblies comprising a spring drive.

FIGS. 22A-22D depict another embodiment of the present fluid delivery assemblies comprising a rotary cam drive.

FIGS. 23-29 depict one example of a prototype (and/or components of the prototype) of the present fluid delivery assemblies.

FIG. 30 depicts a benchmark device used to compare to the prototype of FIGS. 23-29.

### DESCRIPTION OF ILLUSTRATIVE EMBODIMENTS

The term “coupled” is defined as connected, although not necessarily directly, and not necessarily mechanically or electrically. Two items are “couplable” if they can be coupled to each other. Unless the context explicitly requires otherwise, items that are couplable are also decouplable, and vice-versa. One non-limiting way in which a first structure is couplable to a second structure is for the first structure to be configured to be coupled to the second structure.

The terms “a” and “an” are defined as one or more unless this disclosure explicitly requires otherwise.

The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, or a component of a system, that “comprises,” “has,” “includes” or “contains” one or more elements or features possesses those one or more elements or features, but is not limited to possessing only those elements or features. Likewise, a method that “comprises,” “has,” “includes” or “contains” one or more steps possesses those one or more steps, but is not limited to possessing only those one or more steps. Additionally, terms such as “first” and “second” are used only to differentiate structures or features, and not to limit the different structures or features to a particular order.

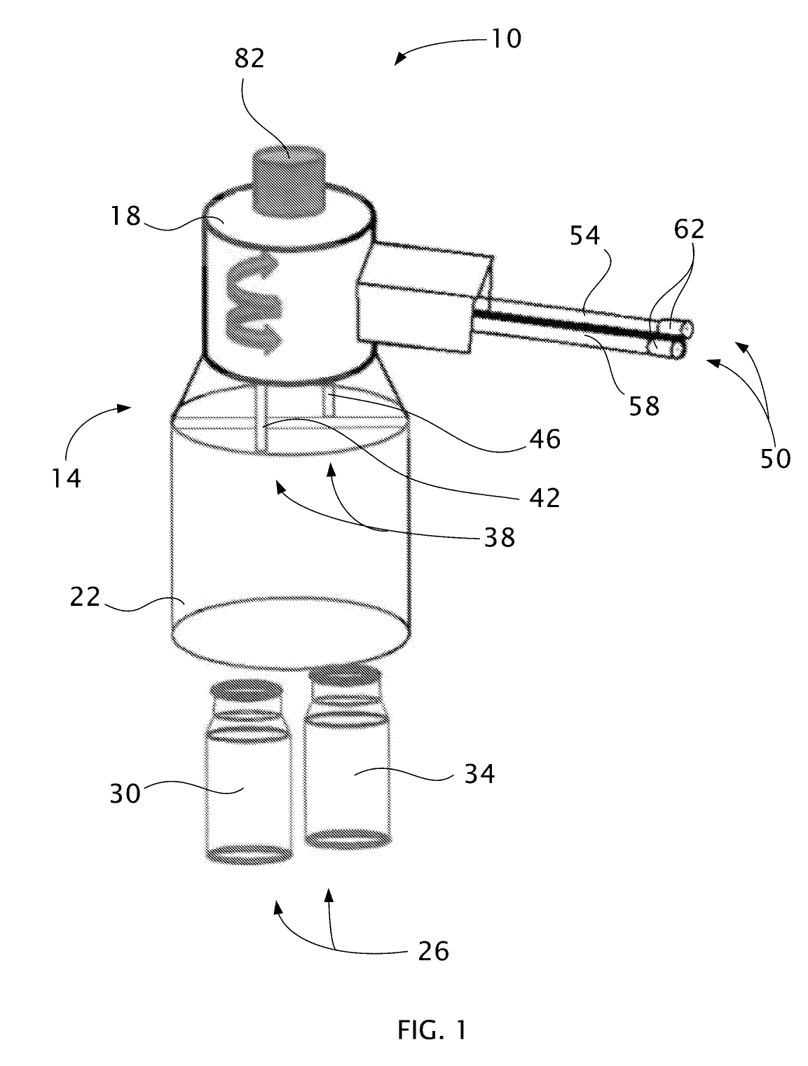

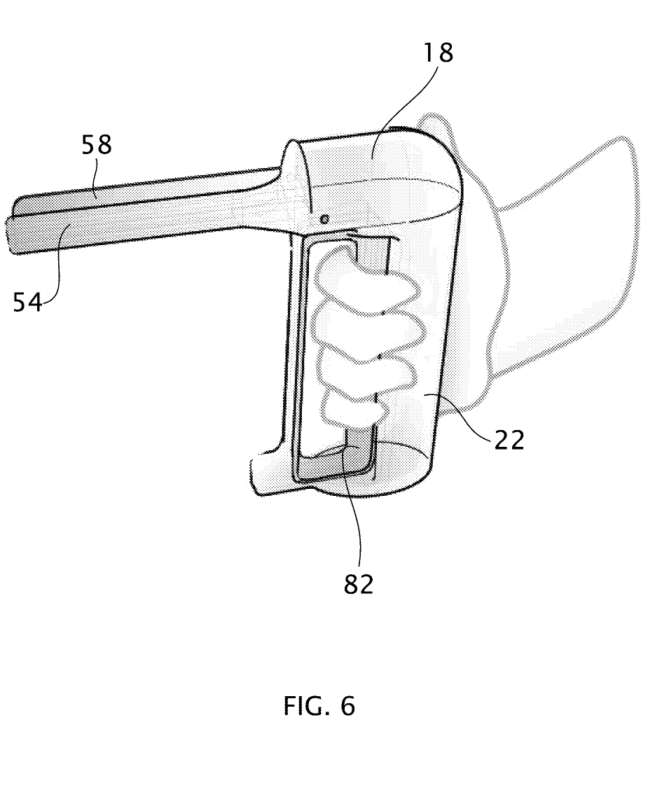

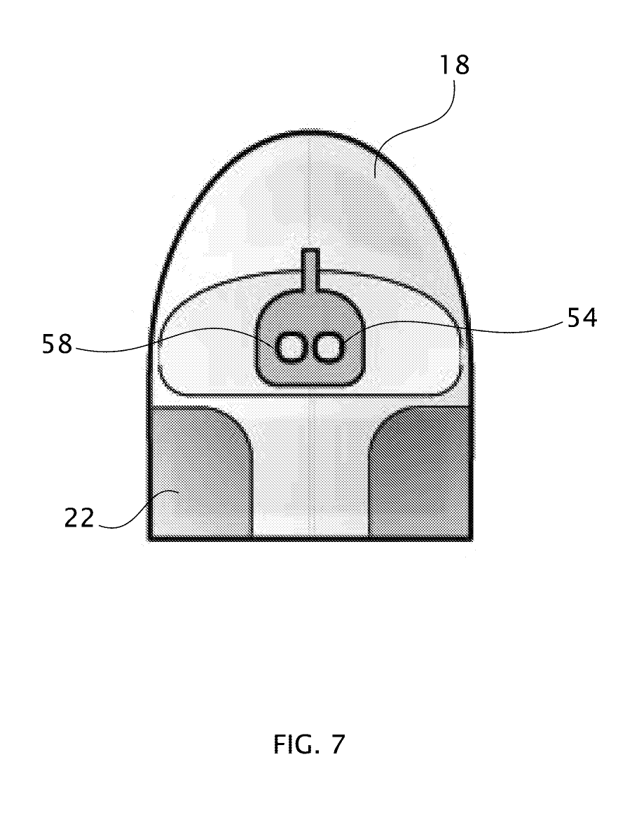

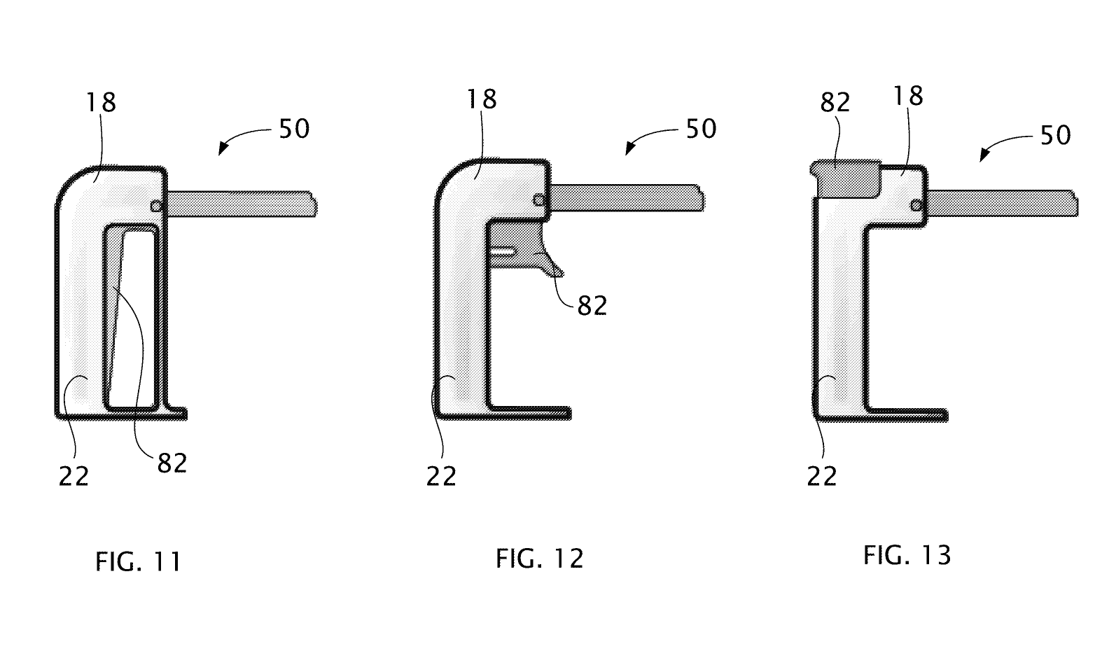

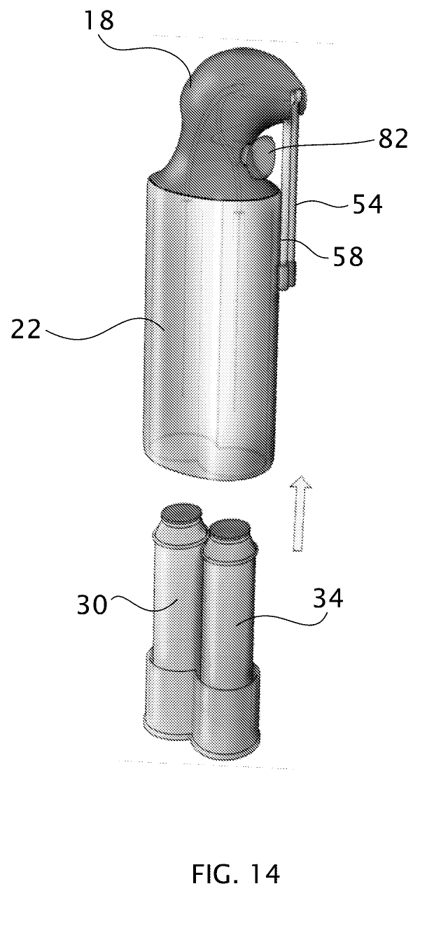

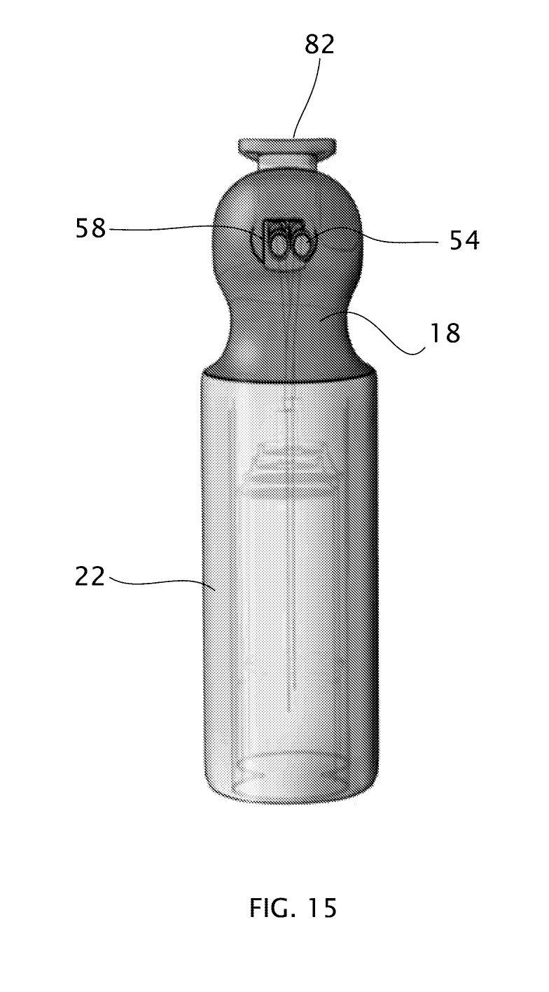

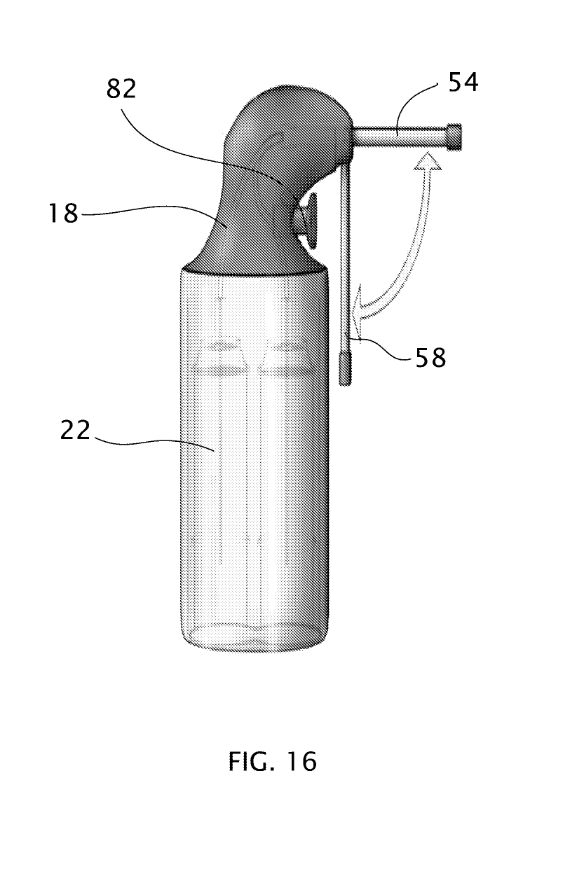

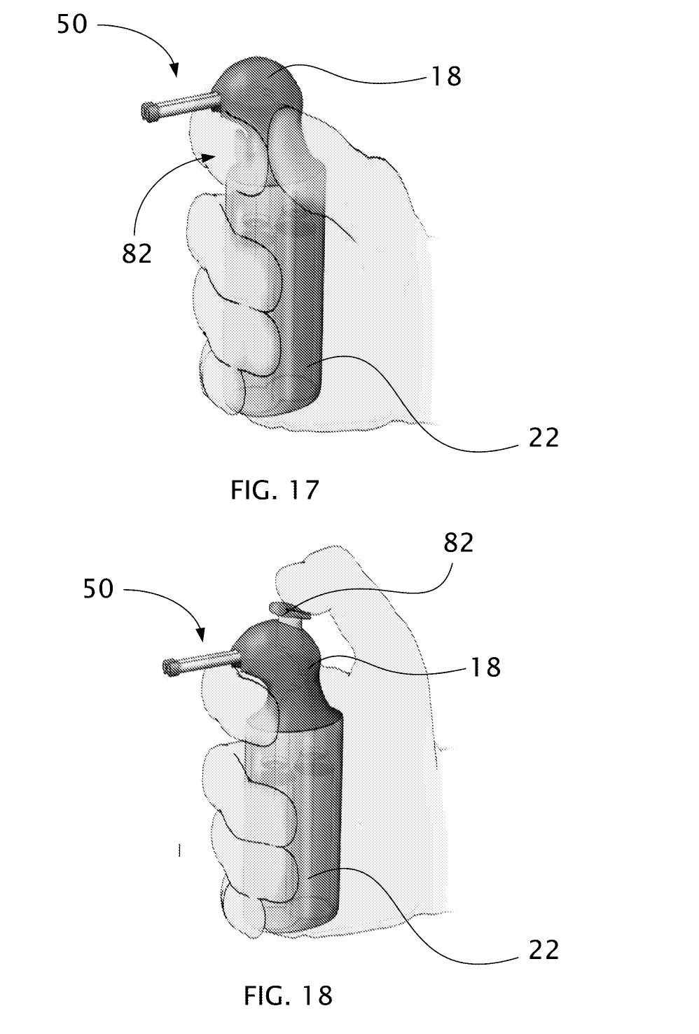

Referring now to FIGS. 1-18, designated by reference numeral 10 are several embodiments of the present fluid delivery assemblies. Fluid delivery assembly 10 comprises body 14. In the embodiments shown, body 14 comprises upper portion 18 and lower portion 22, but is not required to. Upper portion 18 and lower portion 22 can have any suitable configuration. For example, body 14 (or portions of body 14) can have a substantially cylindrical configuration, as depicted in the embodiment shown in FIG. 1. In FIG. 1, upper portion 18 has—but is not required to have—a substantially cylindrical shape that is smaller than a substantially cylindrical shape of lower portion 22 (e.g., upper portion 18 comprises a cylindrical shape with a smaller diameter than the cylindrical shape of lower portion 22). In the embodiments shown in FIGS. 6 and 8A-11, body 14 comprises upper portion 18 (e.g., having and/or coupled to a plurality of outlet channels, discussed further below) and a lower portion 22 (e.g., having and/or coupled to a button, discussed further below). As another example, in the embodiments shown in FIGS. 14-18, body 14 comprises lower portion 22 (e.g., having a similar shape to a corresponding plurality of containers, discussed further below) and upper portion 18 (e.g., having and/or coupled to a button, discussed further below). In the embodiments shown, body 14 is configured to be coupled to plurality of containers 26. Body 14 can comprise any shape configured to accommodate and/or be coupled to plurality of containers 26 (e.g., substantially square, substantially rectangular, etc.). For example, body 14 can be coupled to plurality of containers 26 via threads, adhesives, clamps, and the like, and/or by providing support for plurality of containers 26 such that plurality of containers 26 cannot move away from body 14 (e.g., by closing a bottom of body 14 after plurality of containers 26 are disposed within lower portion 22 of body 14). In the embodiments shown, body 14 is configured to be coupled to first container 30 and second container 34; however, in other embodiments, body 14 can be configured to be coupled to more than two containers (e.g., three, four, five, six, seven, or more containers). Plurality of containers 26 can each contain a fluid (e.g., a solution comprising a suspension of cells, fibrinogen, thrombin, and the like) configured to be delivered to a person's body (e.g., fibrinogen and thrombin can mix at a target area on a person to form fibrin).

In the embodiments shown, assembly 10 further comprises plurality of inlet channels 38 (e.g., inlet channel 42 and inlet channel 46). In other embodiments, assembly 10 can comprise any number of inlet channels configured to correspond to a desired number of containers 26 (e.g., three, four, five, six, seven, or more inlet channels). In the embodiments shown, body 14 is configured to accommodate and/or be coupled to plurality of containers 26 such that an interior of plurality of containers 26 is in fluid communication with an inlet channel of plurality of inlet channels 38.

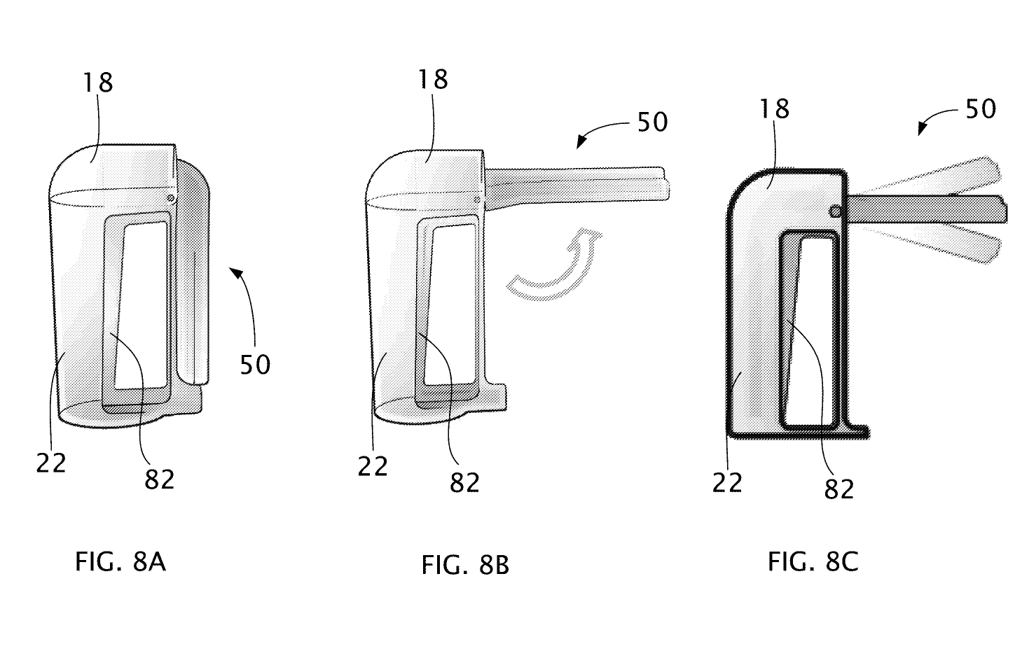

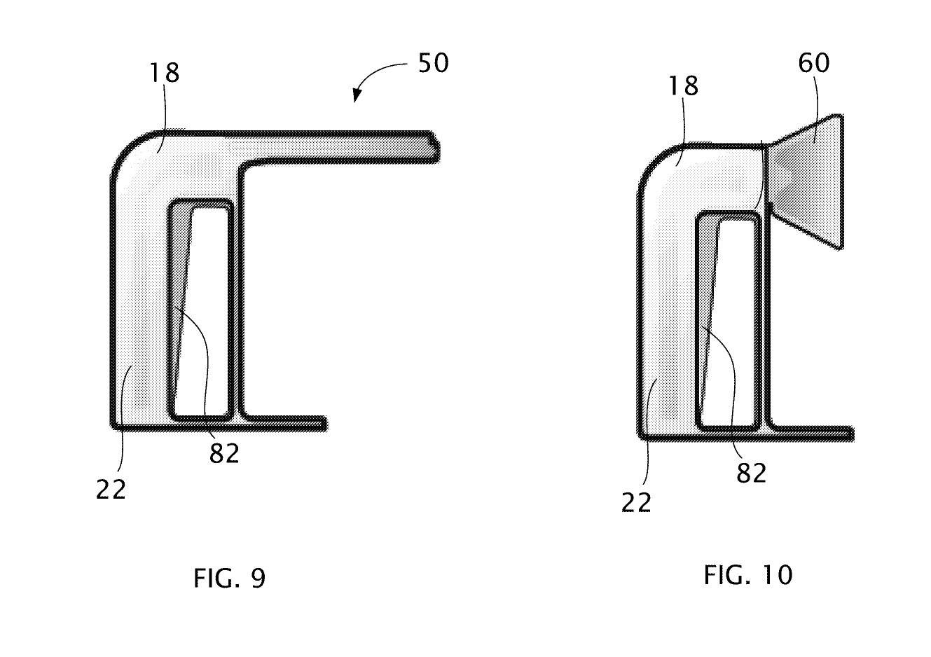

In the embodiments shown, assembly 10 also comprises plurality of outlet channels 50 (e.g., outlet channel 54 and outlet channel 58). Plurality of outlet channels 50 are—but are not required to be—adjacent and/or parallel to one another. In the embodiments shown, plurality of outlet channels 50 extend from body 14, but are not required to. In other embodiments, assembly 10 can comprise any number of outlet channels configured to correspond to a desired number of containers 26 and/or a desired number of inlet channels 38 (e.g., three, four, five, six, seven, or more outlet channels). Each of plurality of outlet channels 50 corresponds to and is configured to be in fluid communication with a corresponding inlet channel of plurality of inlet channels 38 (e.g., such that fluid from each of plurality of containers 26 can pass through a corresponding inlet channel and into a corresponding outlet channel when plurality of containers 26 are coupled to body 14). Plurality of outlet channels 50 can also be configured to atomize a fluid such that fluid from plurality of outlet channels 50 separates into smaller units of fluid (e.g., a spray). For example, each of plurality of outlet channels 50 can comprise nozzle 62. Nozzle 62 can comprise any configuration suitable to atomize a fluid, such as, for example, plain orifice nozzles, shaped orifice nozzles (e.g., comprising a hemispherical shaped inlet and a “V” notched outlet), surface impingement nozzles (e.g., spiral designs), pressure swirl nozzles, solid cone nozzles, compound nozzles, and/or any two-fluid nozzles, if required. Plurality of outlet channels 50 can also be adjustable (e.g., as depicted in FIGS. 8A-C, 14, and 16) or non-adjustable (e.g., as depicted, in FIG. 9). Further, body 14 can comprise various configurations to deliver fluid, such as, for example cone 60 coupled to body 14 around plurality of outlet channels 50 and configured to permit fluid to be delivered to a greater area than in other embodiments.

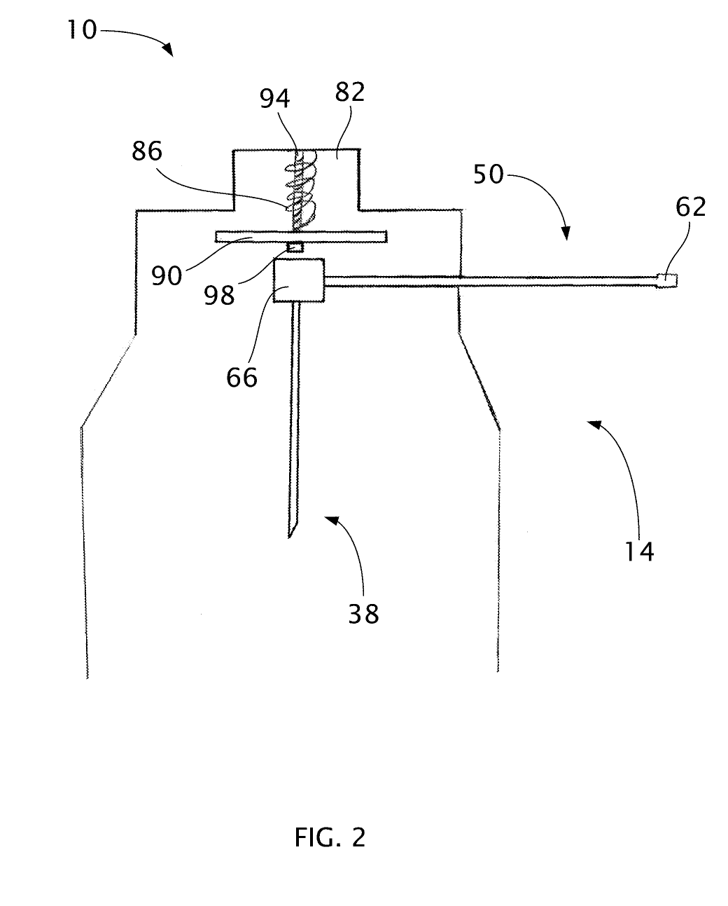

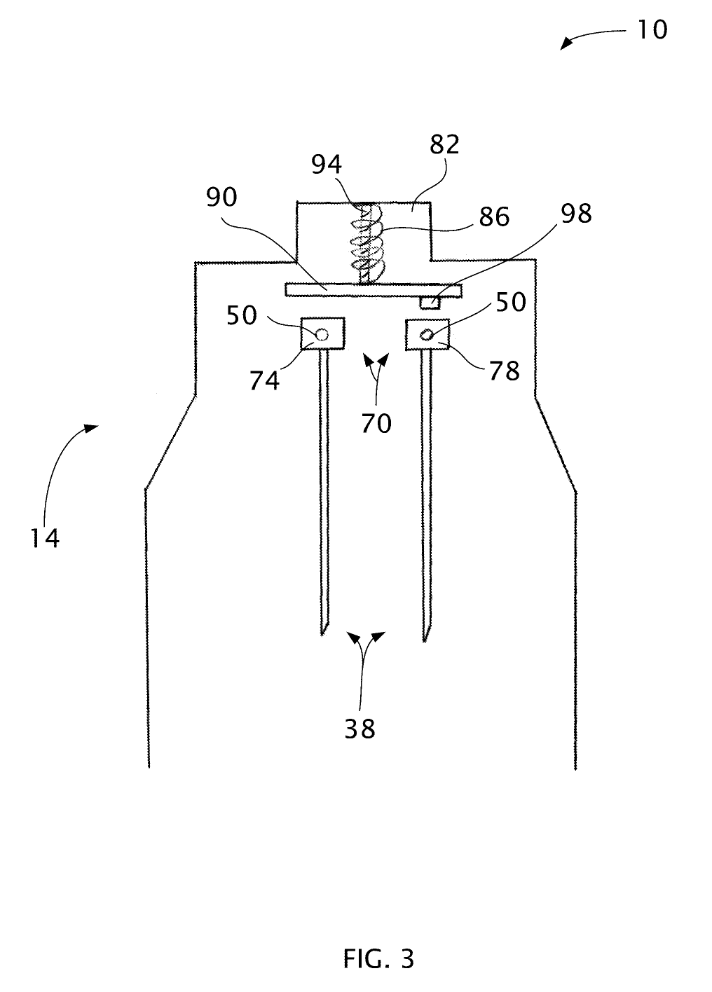

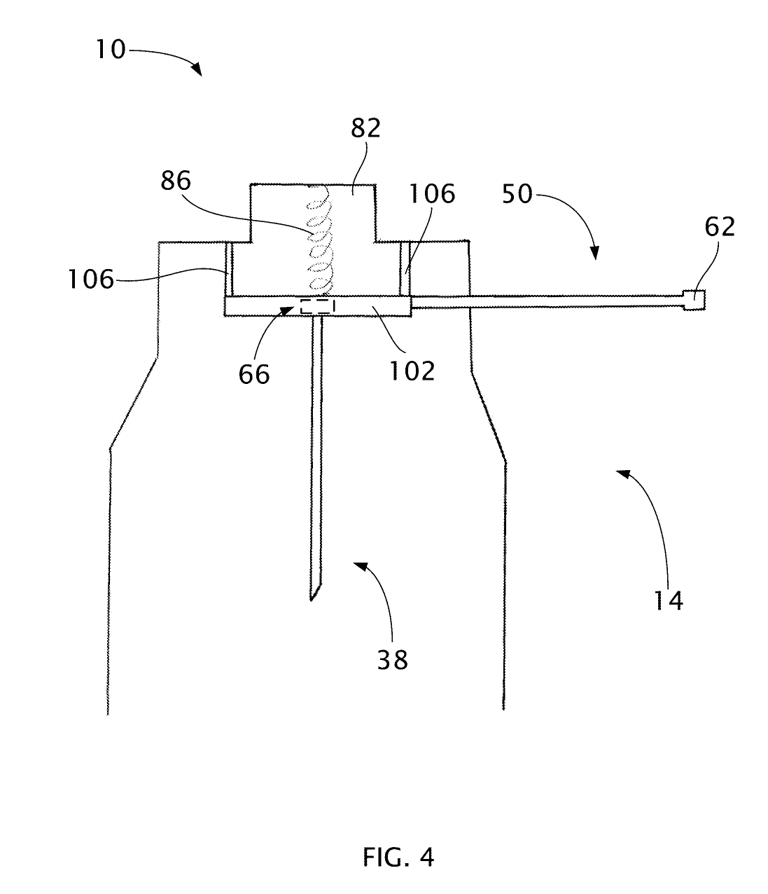

In the embodiments shown, assembly 10 can comprise pump 66 (e.g., as depicted in the embodiment shown in FIG. 4). In some embodiments, assembly 10 can comprise plurality of pumps 70 (e.g., pump 74 and pump 78, as depicted in the embodiment shown in FIGS. 2-3). In other embodiments, plurality of pumps 70 can comprise any number of pumps configured to correspond to plurality of containers 26, plurality of inlet channels 38, and/or plurality of outlet channels 54. Pump 66 and/or plurality of pumps 70 can be coupled (e.g., directly or indirectly) to plurality of inlet channels 38 and/or plurality of outlet channels 50 such that when assembly 10 is actuated, fluid is pumped by pump 66 and/or plurality of pumps 70 (e.g., pump 74 and pump 78) from plurality of containers 26 (e.g., when plurality of containers 26 are coupled to body 14), through plurality of inlet channels 38 and plurality of outlet channels 58, and out of nozzles 62. Pump 66 and/or plurality of pumps 70 can be coupled (e.g., directly or indirectly) to body 14 of assembly 10. In the embodiments shown, assembly 10 (e.g., and pump 66 and/or plurality of pumps 70) is configured to be actuated by pressing button 82 (e.g., applying a force to button 82 in a direction toward body 14). Button 82 can be—but is not required to be—configured to return to an un-pressed configuration after being pressed. For example, in the embodiment shown in FIGS. 2-4, assembly 10 further comprises spring 86 coupled to body 14 (e.g., and more specifically, coupled to button 86). Spring 86 is configured to compress when a force is applied to button 82 in a direction toward body 14. When such a force is released from button 82, spring 86 is configured to relax (e.g., permitting button 82 to return to an un-pressed configuration). Further, button 82 can be coupled to body 14 in any suitable way to permit a user to actuate assembly 10. For example, in the embodiments shown in FIGS. 1, 13, 15, and 18, button 82 is coupled to upper portion 18 of body 14 such that a user can press button 82 toward lower portion 22 to actuate assembly 10. As another example, in the embodiments shown in FIGS. 6 and 8A-11, button 82 is coupled to lower portion 22 such that a user can press button 82 toward lower portion 22 to actuate assembly 10. In yet another example, in the embodiments shown in FIGS. 14, 16, and 17, button 82 is coupled to upper portion 18 such that a user can press button 82 toward upper portion 18 to actuate assembly 10. Button 82 can also be coupled to both upper portion 18 and lower portion 22 (e.g., as depicted in FIG. 12).

In the embodiments shown, assembly 10 is configured to deliver fluid from plurality of containers 26. For example, assembly 10 can be configured to first deliver fluid from container 30 and subsequently deliver fluid from container 34 (or vice versa). In the embodiment shown in FIGS. 2-3, assembly 10 comprises member 90 coupled to body 14 (e.g., via rotating bar 94). Member 90 comprises (or is coupled to) pump actuator 98. For example, if plurality of containers 26 are coupled to body 14, button 82 can be pressed (e.g., by applying a force to button 82 in a direction toward body 14) to actuate one of plurality of pumps 70 (e.g., pump 78). Upon actuation, the one of plurality of pumps 70 (e.g., pump 78) is configured to pump fluid from a corresponding container of plurality of containers 26, through a corresponding inlet channel of plurality of inlet channels 38, and out a corresponding outlet channel of plurality of outlet channels 50 (e.g., via nozzle 62). Rotating bar 94 can then be configured to rotate member 90 (e.g., substantially 180°, in the embodiment shown) such that pump actuator 98 is in a suitable position to actuate another pump of plurality of pumps 70 (e.g., pump 74, in the embodiment shown). Button 82 can be pressed again such that pump actuator 98 can actuate the another of plurality of pumps 70 (e.g., pump 74). Upon actuation, the another of plurality of pumps 70 (e.g., pump 74) is configured to pump fluid from a corresponding container of plurality of containers 26, through a corresponding inlet channel of plurality of inlet channels 38, and out a corresponding outlet channel of plurality of outlet channels 50 (e.g., via nozzle 62). In such an embodiment, fluid from plurality of containers 26 (e.g., a first fluid and a second fluid) is pumped with successive actuations of assembly 10 (e.g., successively pressing button 82). In other embodiments, assembly 10 can be configured such that fluid from plurality of containers 26 (e.g., a first fluid and a second fluid) is pumped with one actuation of the assembly (e.g., pressing button 82 once such that the assembly successively actuates each of plurality of pumps 70 (e.g., pump 74 and pump 78, in the embodiment shown)).

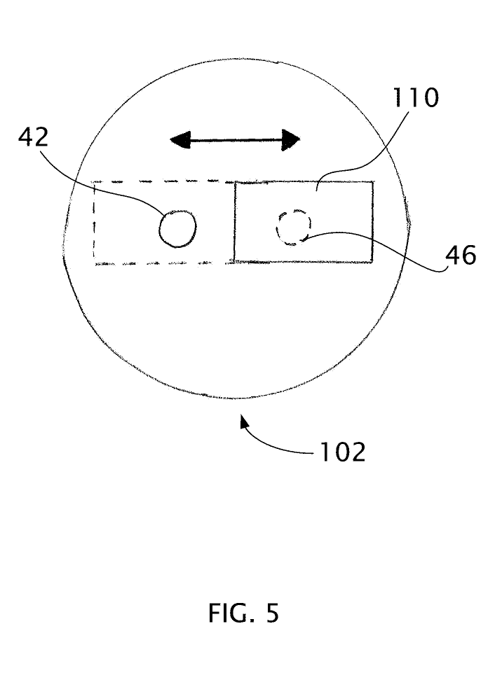

The embodiment shown in FIGS. 4-5 depicts another embodiment in which assembly 10 is configured to deliver fluid from plurality of containers 26. For example, assembly 10 can similarly be configured to first deliver fluid from container 30 and subsequently deliver fluid from container 34 (or vice versa). In the embodiment shown in FIGS. 4-5, assembly 10 comprises at least one valve 102. At least one valve 102 can be coupled to body 14 (e.g., via support members 106), plurality of inlet channels 38, and/or plurality of outlet channels 50. At least one valve 102 can comprise sliding member 110. In the embodiment shown, at least one valve 102 is configured to be actuated between a plurality of configurations, each configuration permitting fluid communication between one of plurality of inlet channels 38 (e.g., inlet channel 42) and one of plurality of outlet channels 50 (e.g., outlet channel 58), and each configuration preventing fluid communication between the other(s) of plurality of inlet channels 38 (e.g., inlet channel 46) and the other(s) of plurality of outlet channels 50 (e.g., outlet channel 54). For example, at least one valve 102 is configured to be actuated between a first configuration and a second configuration. The first configuration of at least one valve 102 can be configured to permit fluid communication between inlet channel 42 and outlet channel 58 and to prevent fluid communication between inlet channel 46 and outlet channel 54. The second configuration of at least one valve 102 can be configured to permit fluid communication between inlet channel 46 and outlet channel 54 and to prevent fluid communication between inlet channel 42 and outlet channel 58. In the first configuration, assembly 10 can be actuated (e.g., by pressing button 82) to pump (e.g., with at least one pump 66) a fluid from one of plurality of containers 26 (e.g., container 30), through inlet channel 42 and at least one valve 102, and out outlet channel 58 (e.g., via nozzle 62). In the second configuration, assembly 10 can be actuated (e.g., by pressing button 82) to pump (e.g., with at least one pump 66) a fluid from another of plurality of containers 26 (e.g., container 34), through inlet channel 46 and at least one valve 102, and out outlet channel 54 (e.g., via nozzle 62). In some embodiments, at least one pump 66 and/or button 82 is coupled to at least one valve 102 such that at least one valve 102 actuates between the plurality of configurations (e.g., the first and second configuration) with successive actuations of assembly 10 (e.g., two actuations, in the embodiment shown). In other embodiments, at least one pump 66 and/or button 82 is coupled to at least one valve 102 such that at least one valve 102 alternates between the plurality of configurations (e.g., the first and second configuration) with one actuation of assembly 10.

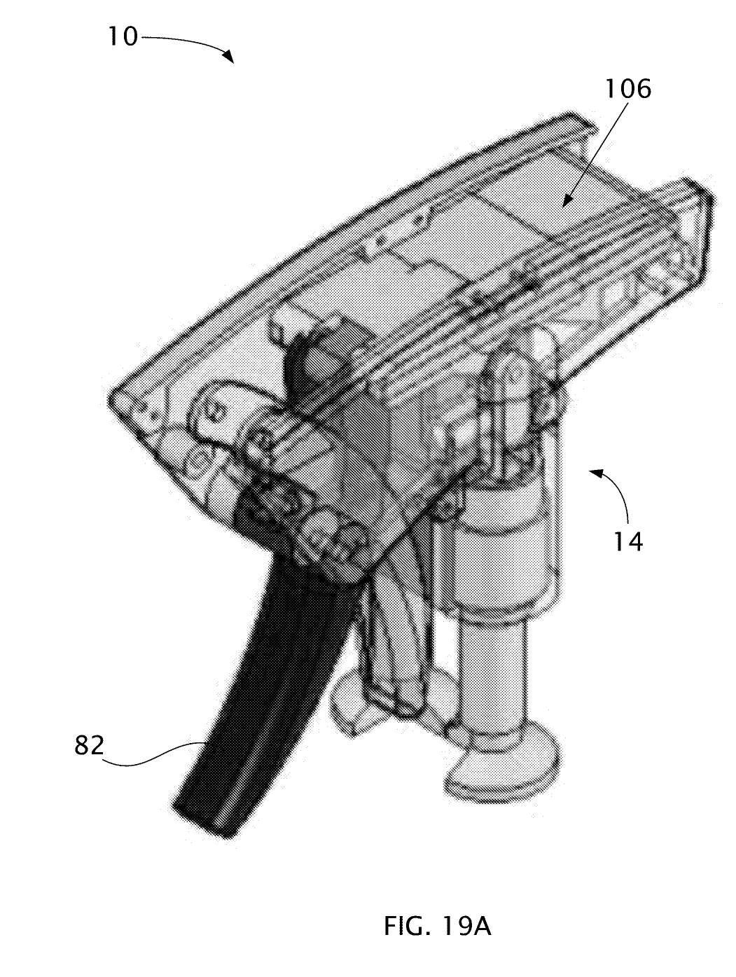

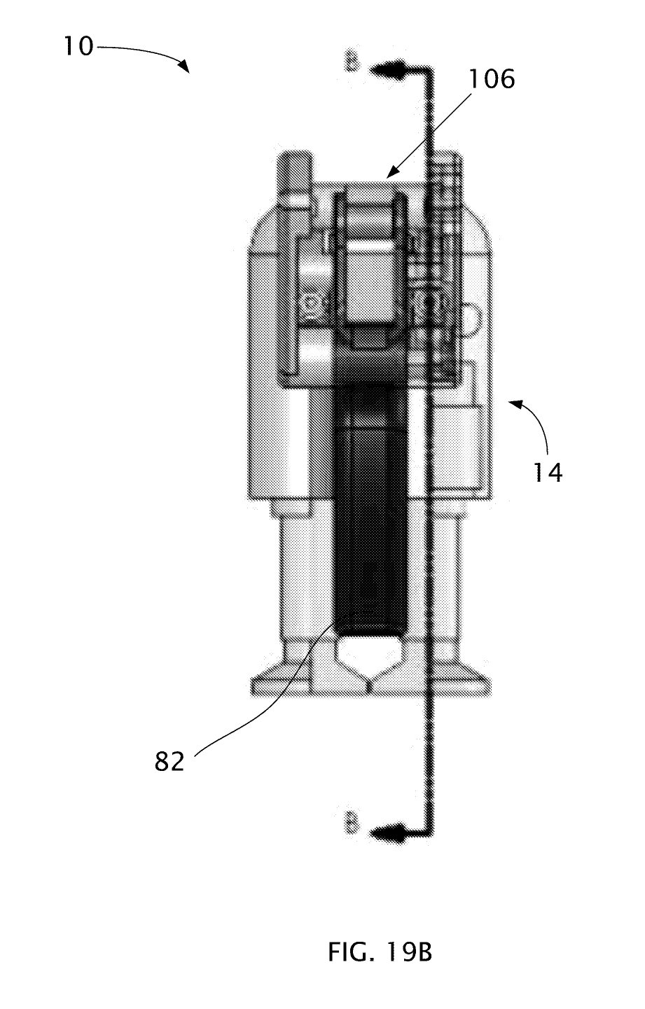

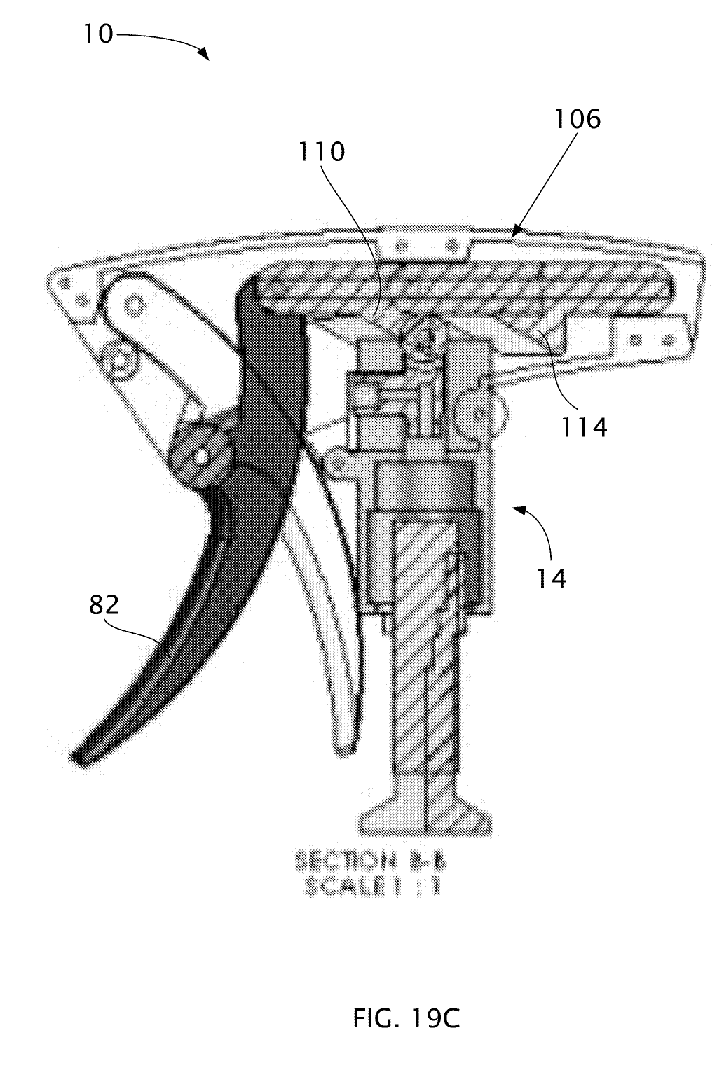

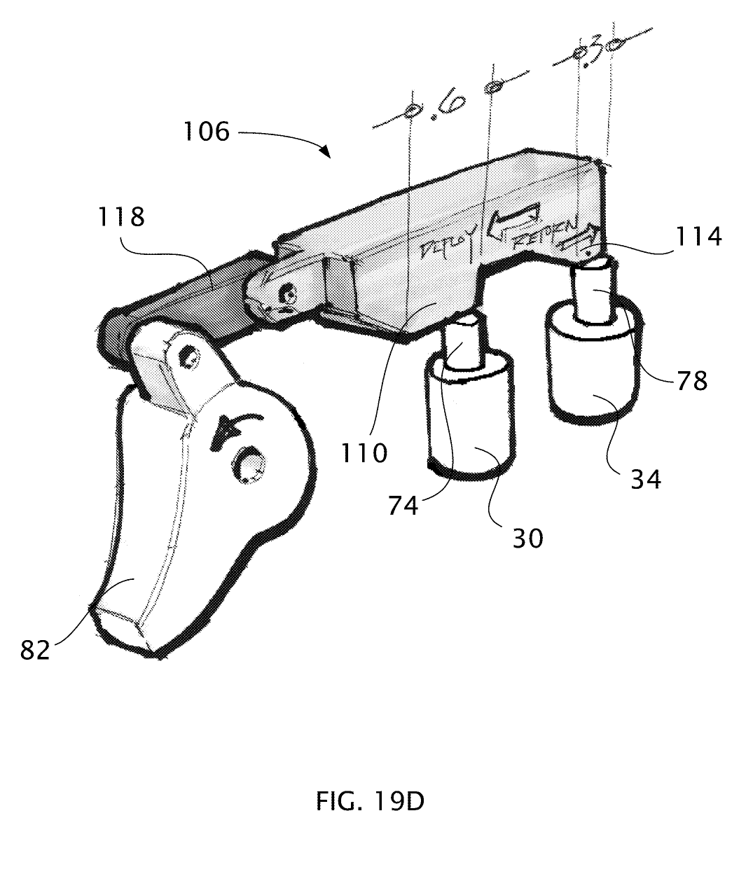

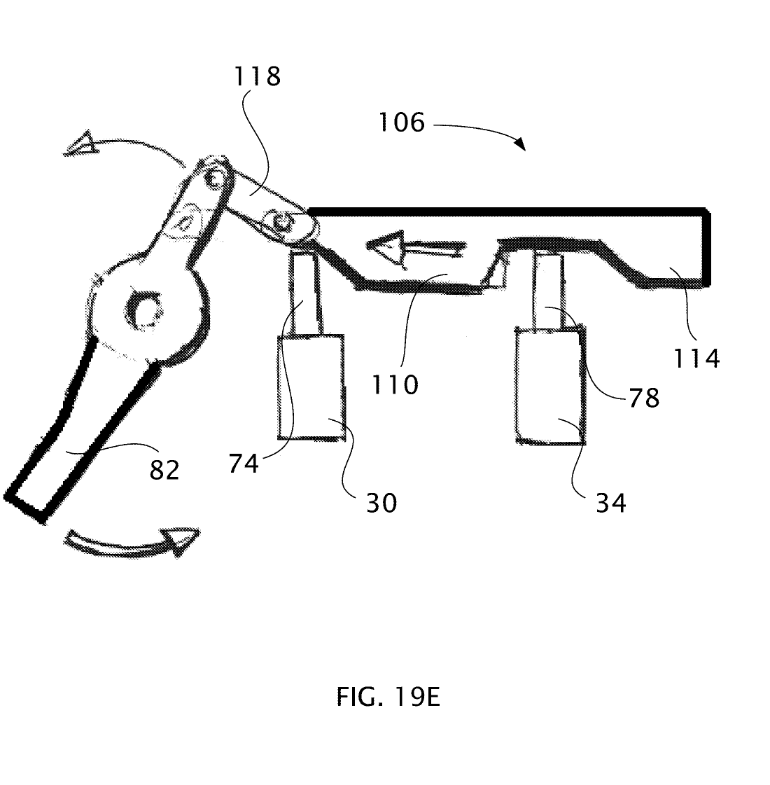

FIGS. 19A-19E depict another embodiment of assembly 10 (or components of assembly 10) configured, upon at least one actuation (e.g., one actuation, in the embodiment shown), to successively pump fluid from, for example, container 30 (e.g., through a first inlet channel and out of a first outlet channel) and subsequently pump fluid from container 34 (e.g., through a second inlet channel and out of a second outlet channel), or vice versa. In the embodiment shown, assembly 10 comprises linear cam drive 106, which comprises cam 110 and cam 114. Linear cam drive 106 is coupled to button 82 (e.g., having a trigger-like configuration, in the embodiment shown) by member 118. For example, if container 30 and container 34 are coupled to assembly 10, button 82 can be pressed (e.g., by applying a force to button 82 in a direction toward body 14) such that linear cam drive 106 moves toward button 82 to actuate pump 74 with cam 110. Upon actuation, pump 74 is configured to pump fluid from container 30, through a corresponding inlet channel, and out of a corresponding outlet channel. Subsequent to the actuation of pump 74, in the embodiment shown, linear cam drive 106, which continues to move toward button 82, actuates pump 78 with cam 114. Upon actuation, pump 78 is configured to pump fluid from container 34, through a corresponding inlet channel, and out of a corresponding outlet channel. Button 82 can be—but is not required to be—configured to return to an un-pressed configuration after being pressed. For example, assembly 10 can include a spring that is configured to compress when a force is applied to button 82 in a direction toward body 14; and, if such a force is released from button 82, the spring is configured to relax (e.g., permitting button 82 to return to an un-pressed configuration).

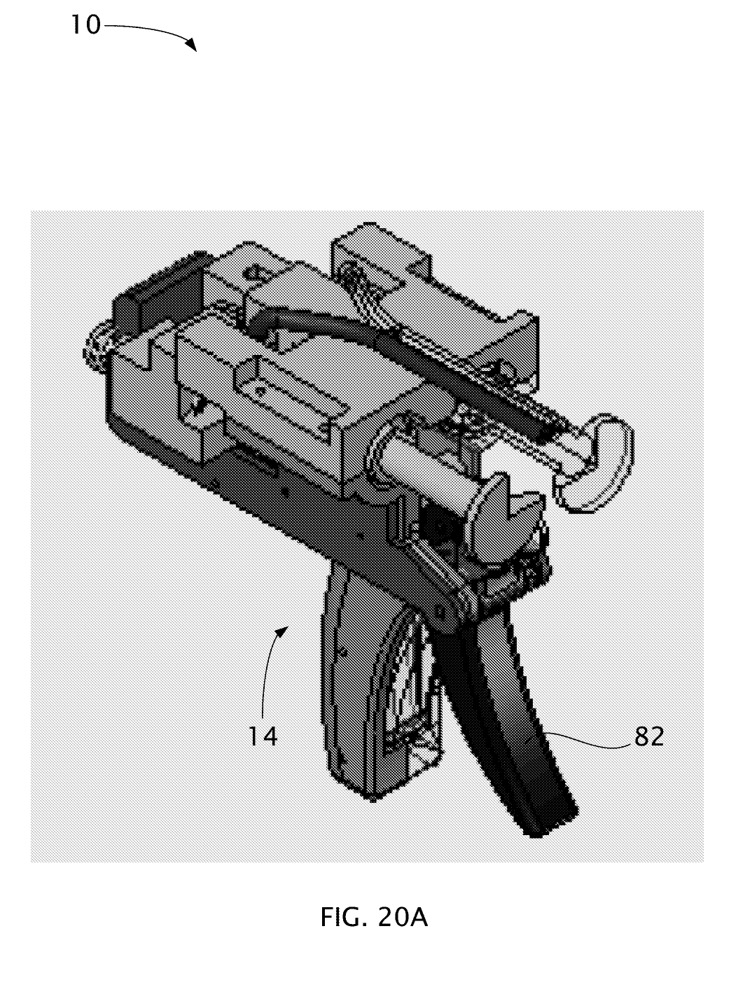

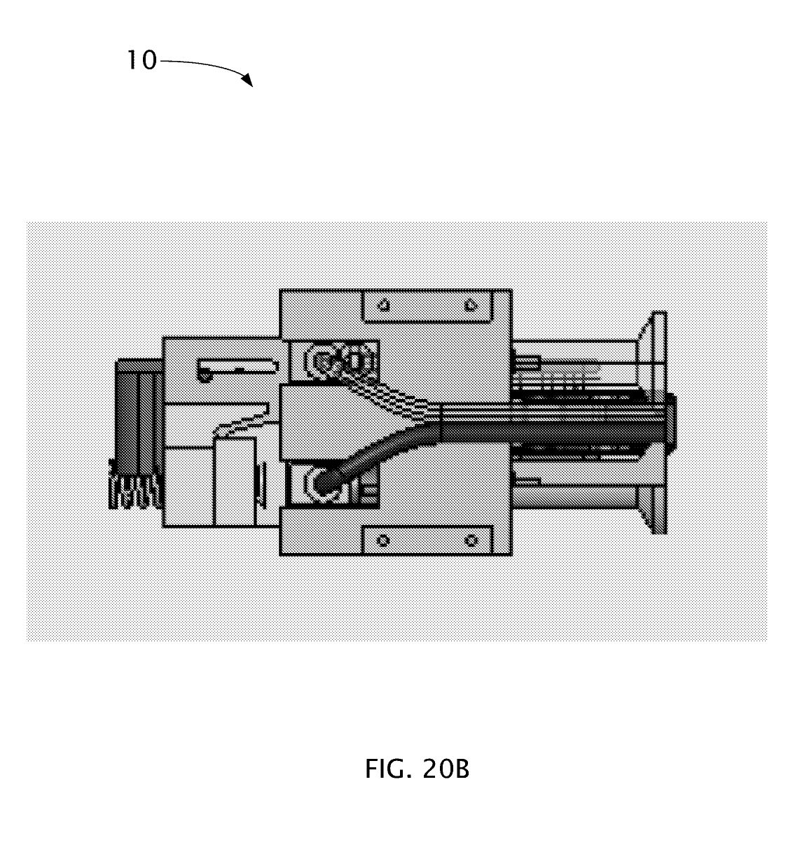

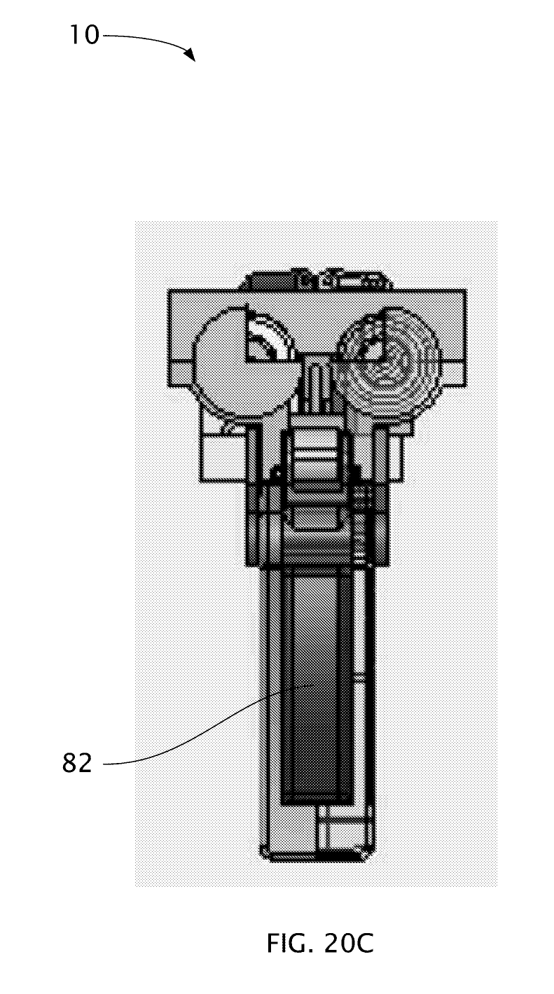

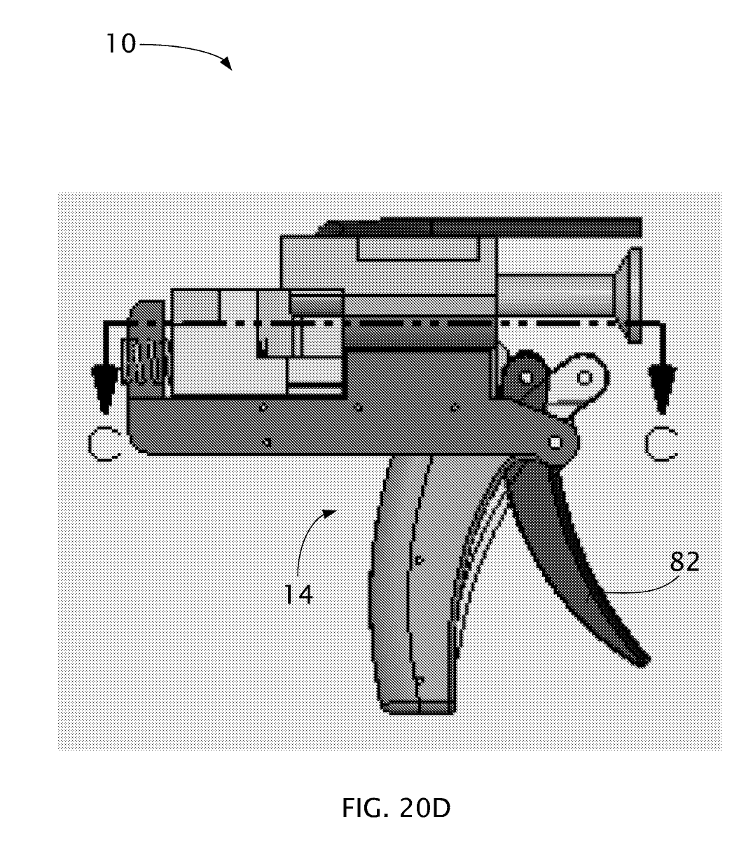

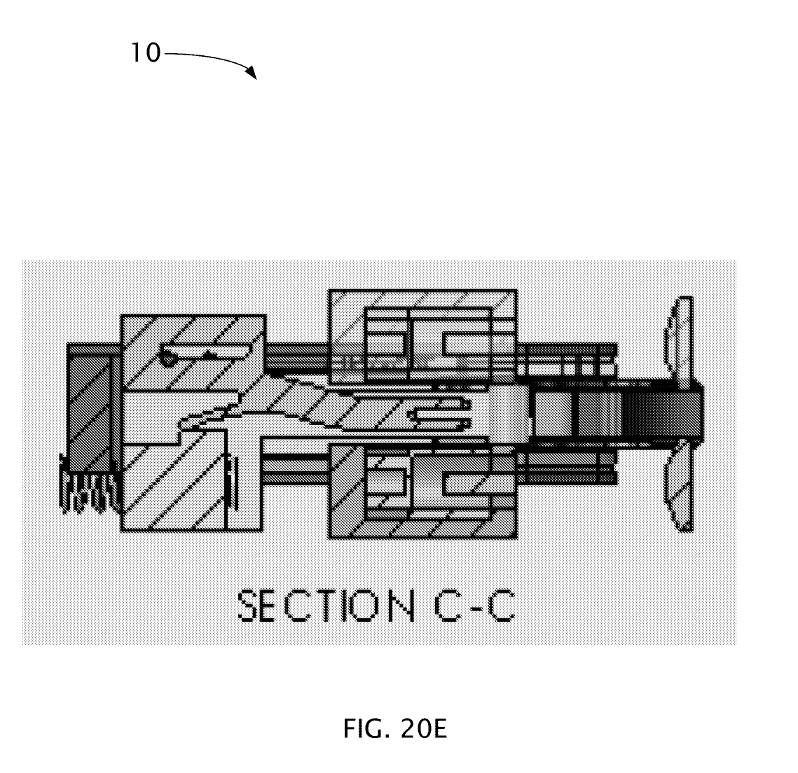

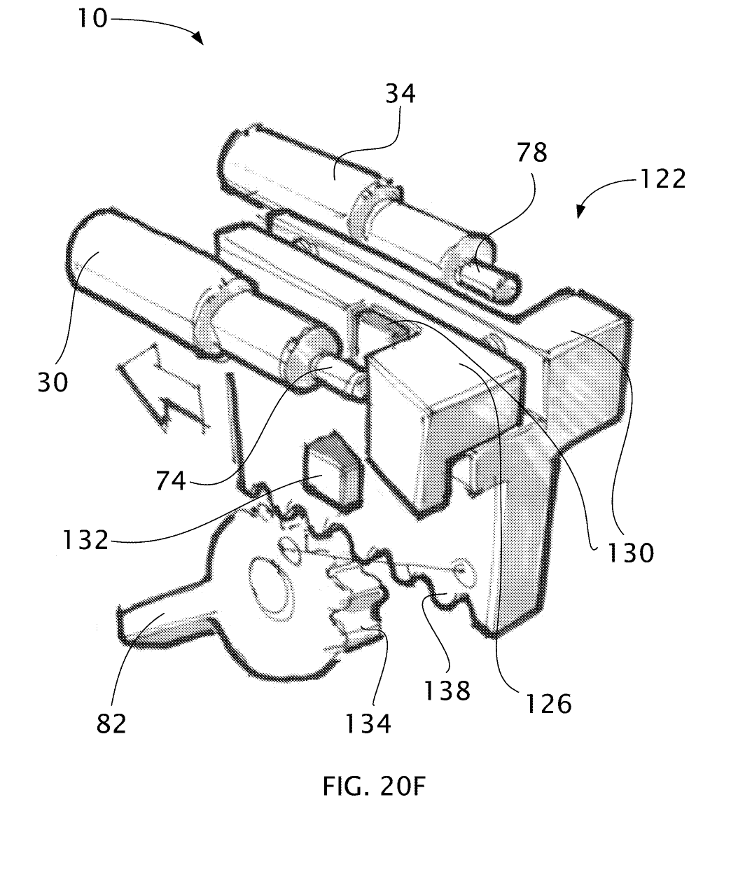

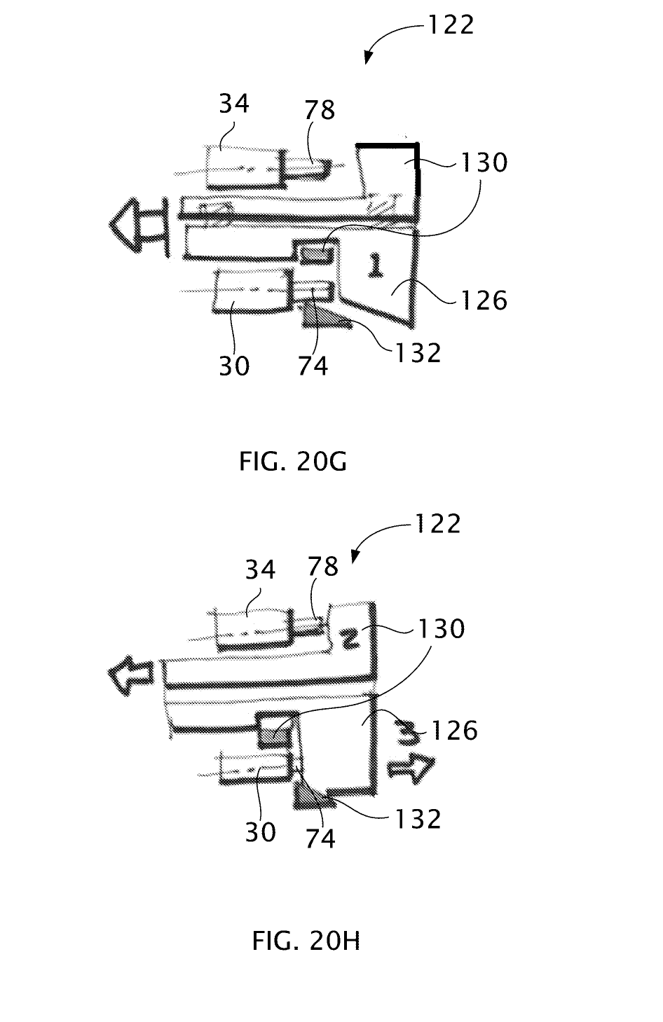

FIGS. 20A-20I depict another embodiment of assembly 10 (or components of assembly 10) configured, upon at least one actuation (e.g., one actuation, in the embodiment shown), to successively pump fluid from, for example, container 30 (e.g., through a first inlet channel and out of a first outlet channel) and subsequently pump fluid from container 34 (e.g., through a second inlet channel and out of a second outlet channel), or vice versa. In the embodiment shown, assembly 10 comprises rack drive 122, which comprises rack member 126 and rack member 130. Rack drive 122 is coupled to button 82 (e.g., having a trigger-like configuration, in the embodiment shown) by threads. In the embodiment shown, button 82 comprises threads 134, and rack drive 122 comprises threads 138. If button 82 is pressed, threads 134 of button 82 engage threads 138 of rack drive 122 such that rack drive 122 (e.g., and more specifically, rack member 126 and rack member 130) move toward container 30 and container 34 to actuate pump 74 and pump 78. In the embodiment shown in FIGS. 20E-20H, rack member 130 of rack drive 122 comprises threads 138 configured to engage threads 134 of button 82, and rack member 126 of rack drive 122 is coupled to rack member 130 such that if rack member 130 moves, rack member 126 moves with rack member 130. In the embodiment shown in FIG. 20I, both rack member 126 and rack member 130 of rack drive 122 comprise threads 138 configured to engage threads 134 of button 82 such that if button 82 moves, threads 138 of each of rack member 126 and rack member 130 engage threads 134 of button 82 such that rack member 126 and rack member 130 move. For example, if container 30 and container 34 are coupled to assembly 10, button 82 can be pressed (e.g., by applying a force to button 82 in a direction toward body 14) such that rack drive 122 (and, more specifically, rack member 130 and rack member 126 (e.g., because rack member 126 is coupled to rack member 130 as depicted in FIGS. 20E-20H, because rack member 126 comprises threads 138 that are engaged with threads 134 of button 82, as depicted in FIG. 20I, and the like)) moves toward container 30 and container 34 and actuates pump 74 (corresponding to container 30). Upon actuation of pump 74 by rack drive 122 (and, more specifically, rack member 126), pump 74 is configured to pump fluid from container 30, through a corresponding inlet channel, and out of a corresponding outlet channel. Subsequent to the actuation of pump 74, rack drive 122 (and, more specifically, rack member 130 and rack member 126) continues to move toward container 34 to actuate pump 78 (corresponding to container 34). In the embodiment shown in FIGS. 20E-20H, rack drive 122 further comprises stopper 132, which is configured to prevent rack member 126 from moving toward container 30 after pump 74 is actuated. For example, in the embodiment shown in FIGS. 20E-20H, prior to actuation of pump 78, rack drive 122 is configured to contact stopper 132, which decouples rack member 126 from rack member 130 (e.g., by moving rack member 126 in a direction substantially perpendicular to the direction of motion of rack member 130). In such an embodiment, rack member 130 continues to move toward container 34 while rack member 126 does not continue to move toward container 130). Upon actuation of pump 78 by rack drive 122 (and, more specifically, rack member 130), pump 78 is configured to pump fluid from container 34, through a corresponding inlet channel, and out of a corresponding outlet channel. Button 82 can be—but is not required to be—configured to return to an un-pressed configuration after being pressed. For example, assembly 10 can include a spring that is configured to compress when a force is applied to rack drive 122 by button 82; and, if such a force is released, the spring is configured to relax permitting rack drive 122 to return to its original position and button 82 to return to an un-pressed configuration).

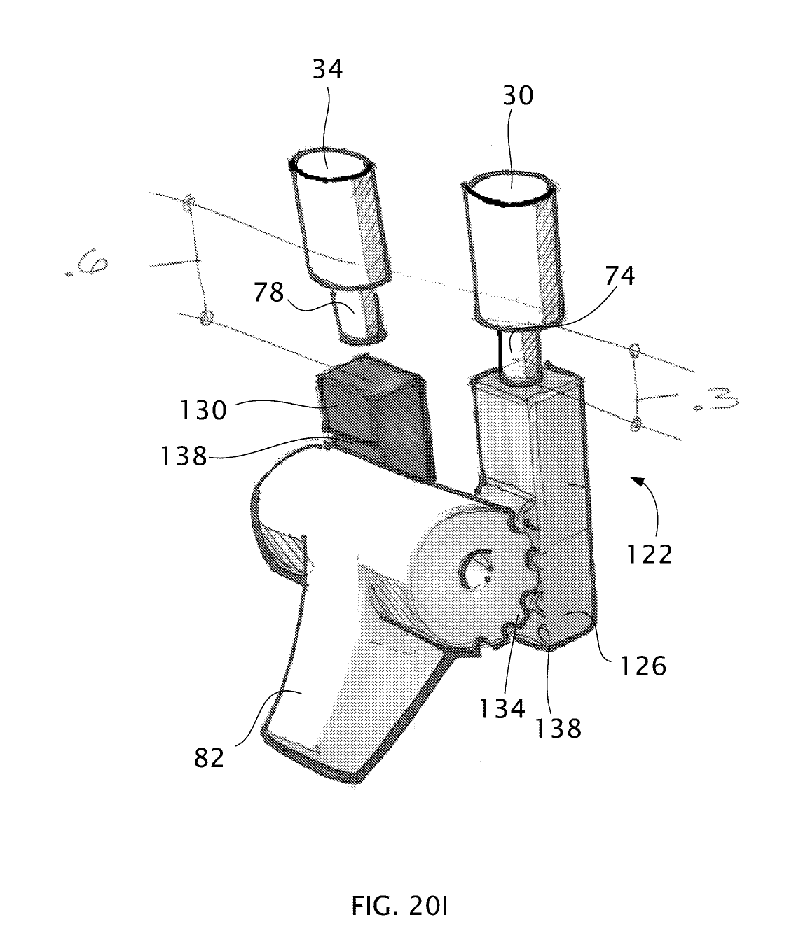

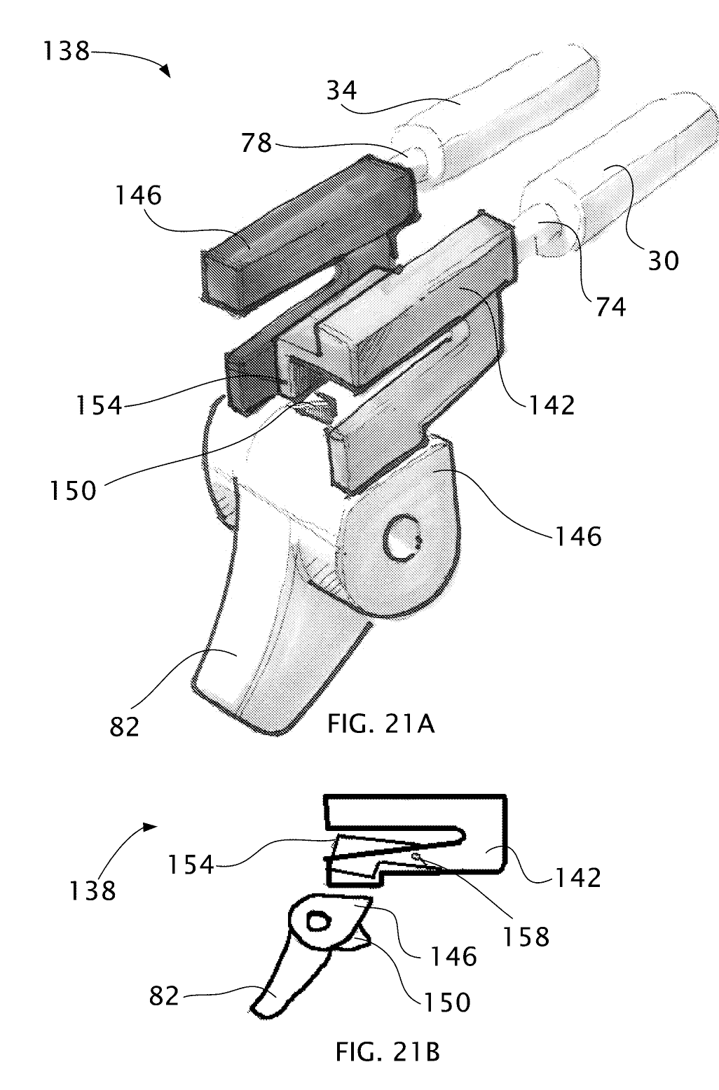

FIGS. 21A-21B depict another embodiment of a drive system that can be used with the present assemblies that is configured, upon at least one actuation (e.g., one actuation, in the embodiment shown), to successively pump fluid from, for example, container 30 (e.g., through a first inlet channel and out of a first outlet channel) and subsequently pump fluid from container 34 (e.g., through a second inlet channel and out of a second outlet channel), or vice versa. In the embodiment shown, assembly 10 comprises spring drive 138, which comprises flex member 142 and flex member 146. In the embodiment shown in FIGS. 21A-21B, button 82 (e.g., having a trigger-like configuration, in the embodiment shown) comprises and/or is coupled to cam 146 and cam 150, which are each configured to engage spring drive 138. Cam 146 and cam 150 are oriented in a staggered configuration such that, if button 82 is pressed (e.g., and cam 146 and cam 150 begin to rotate), a non-cylindrical portion of cam 146 engages spring drive 138 (e.g., and, more specifically, flex member 142) before a non-cylindrical portion of cam 150 engages spring drive 138 (e.g., and, more specifically, flex member 146). Spring drive 138 further comprises lock 154, which is configured to prevent cam 150 from prematurely engaging flex member 146. For example, if button 82 is pressed, cam 146 and cam 150 begin to rotate toward spring drive 138. In the embodiment shown, cam 146 first engages flex member 142, which pivots about flex latch 158, such that flex member 142 actuates pump 74. Upon actuation of pump 74 by flex member 142, pump 74 is configured to pump fluid from container 30, through a corresponding inlet channel, and out of a corresponding outlet channel. Subsequent to the actuation of pump 74, cam 150 engages flex member 146, which pivots about flex latch 158, such that flex member 146 actuates pump 78. Upon actuation of pump 78 by flex member 146, pump 78 is configured to pump fluid from container 34, through a corresponding inlet channel, and out of a corresponding outlet channel. Button 82 can be—but is not required to be—configured to return to an un-pressed configuration after being pressed, as described in detail above, for example, with a spring.

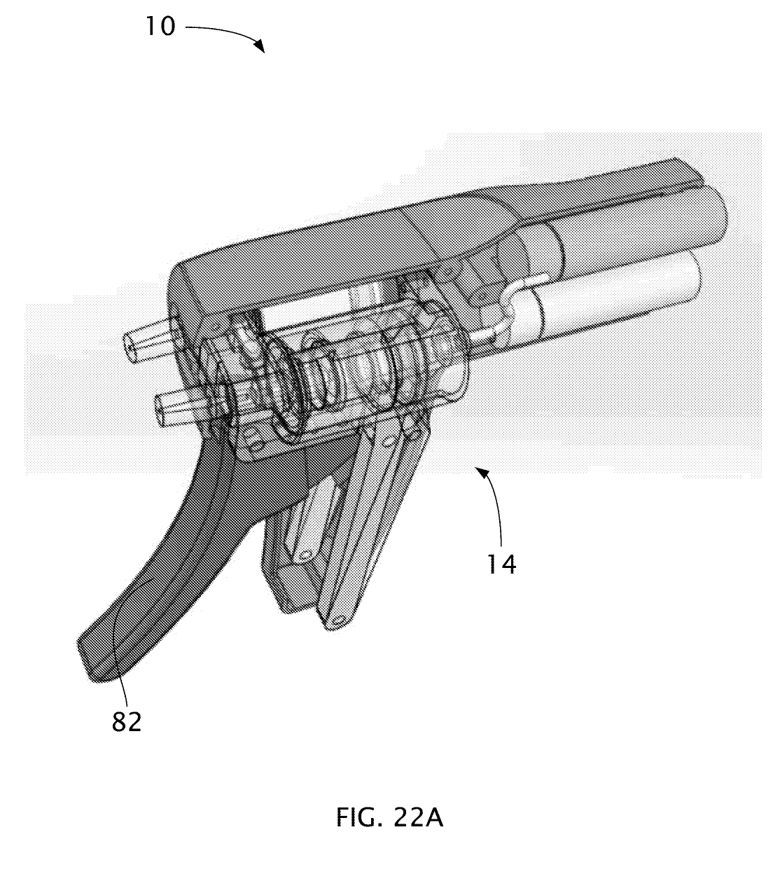

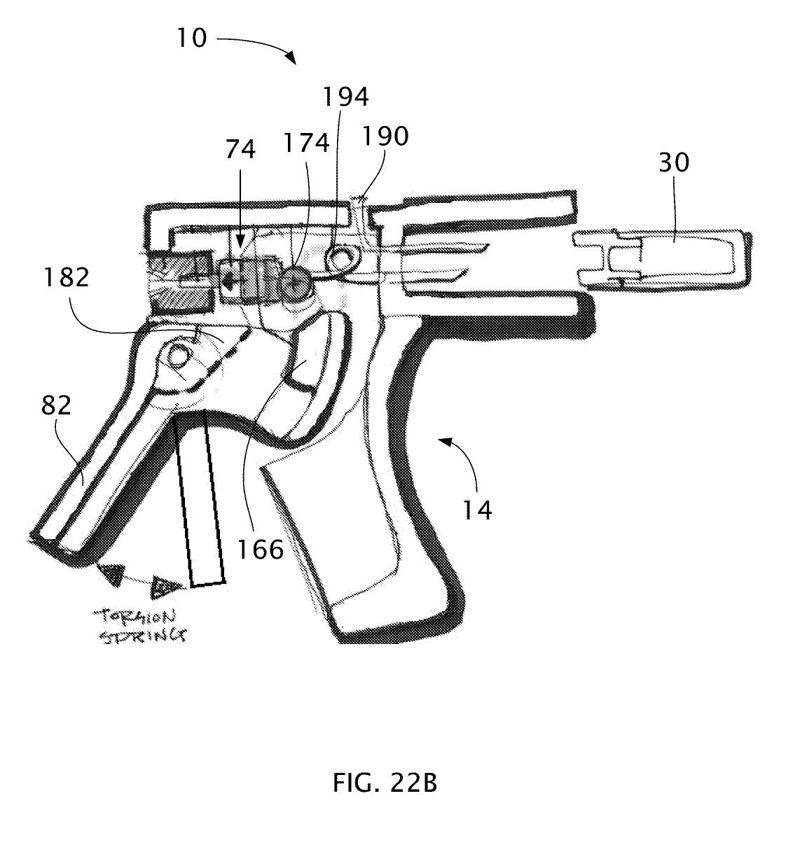

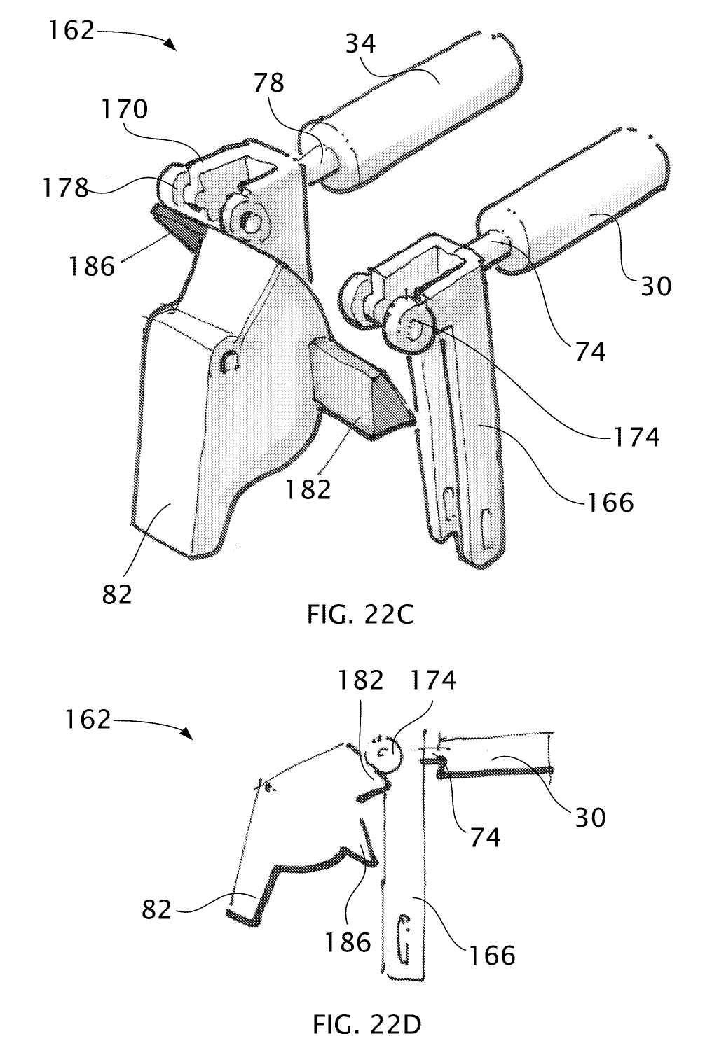

FIGS. 22A-22D depict another embodiment of assembly 10 (or components of assembly 10) configured, upon at least one actuation (e.g., one actuation, in the embodiment shown), to successively pump fluid from, for example, container 30 (e.g., through a first inlet channel and out of a first outlet channel) and subsequently pump fluid from container 34 (e.g., through a second inlet channel and out of a second outlet channel), or vice versa. In the embodiment shown, assembly 10 comprises rotary cam drive 162, which comprises lever 166, lever 170, roller 174, and roller 178. In the embodiment shown, button 82 (e.g., having a trigger-like configuration, in the embodiment shown) comprises and/or is coupled to cam 182 and cam 186, which are each configured to engage rotary cam drive 162. In the embodiment shown in FIG. 22B, assembly 10 further comprises air inlet 190 and flex fluid inlet 194. Air inlet 190 and flex fluid inlet 194 can comprise sharp tips, which can pierce container 30 and/or container 34 if container 30 and/or container 34 are coupled to assembly 10. In the embodiment shown, if container 30 and/or container 34 is coupled to assembly 10 and pierced by air inlet 190 and/or flex fluid inlet 194, air inlet 190 is in fluid communication with the atmosphere and flex fluid inlet 194 is in fluid communication with pump 74. Cam 182 and cam 186 are oriented in a staggered configuration such that, if button 82 is pressed (e.g., and cam 182 and cam 186 begin to rotate), cam 182 engages rotary cam drive 162 (e.g., and, more specifically, lever 166 via roller 174) before cam 186 engages rotary cam drive 162 (e.g., and, more specifically, lever 170 via roller 178). For example, if button 82 is pressed, cam 182 and cam 186 begin to rotate toward rotary cam drive 162 (e.g., and, more specifically, toward roller 174 and roller 178). In the embodiment shown, cam 182 first engages roller 174 and lever 166, which actuates pump 74. Upon actuation of pump 74, pump 74 is configured to pump fluid from container 30, through a corresponding inlet channel, and out of a corresponding outlet channel. Subsequent to the actuation of pump 74, cam 186 engages roller 178 and lever 170, which actuates pump 78. Upon actuation of pump 78, pump 78 is configured to pump fluid from container 34, through a corresponding inlet channel, and out of a corresponding outlet channel. Button 82 can be—but is not required to be—configured to return to an un-pressed configuration after being pressed, as described in detail above, for example, with a spring.

The present disclosure also includes methods of delivering fluid to a person's body. For example, in some embodiments, such methods comprise coupling a plurality of containers (e.g. plurality of containers 26) containing fluid to a fluid delivery assembly (e.g., assembly 10), where the fluid delivery assembly comprises a plurality of inlet channels (e.g., plurality of inlet channels 38), a plurality of outlet channels (e.g., plurality of outlet channels 50), at least one valve (e.g., valve 102), where the at least one valve is configured to alternate between a plurality of configurations, each configuration permitting fluid communication between one of the plurality of inlet channels (e.g., inlet channel 42) and one of the plurality of outlet channels (e.g., outlet channel 58) and preventing fluid communication between the other(s) of the plurality of inlet channels (e.g., inlet channel 46) and the other(s) of the plurality of outlet channels (e.g., outlet channel 54), and at least one pump (e.g., pump 66). Such a method further comprises actuating the pump to successively pump from each of the plurality of containers, through a corresponding inlet channel, and out of an outlet channel. The at least one valve can be configured to alternate between the plurality of configurations with successive actuations (e.g., pressing button 82 a number of times equal to the number of containers from which fluid is pumped) of the assembly and with one actuation of the assembly (e.g., pressing button 82 once).

As another example, the present methods comprise coupling a plurality of containers (e.g., plurality of containers 26) containing fluid to a fluid delivery assembly (e.g., assembly 10), where the fluid delivery assembly comprises a plurality of inlet channels (e.g., plurality of inlet channels 38), a plurality of outlet channels (e.g., plurality of outlet channels 50), and a plurality of pumps (e.g., plurality of pumps 70). Such a method further comprises actuating the plurality of pumps to successively pump from each of the plurality of containers, through a corresponding inlet channel, and out of an outlet channel. The plurality of pumps can be configured to pump from each of the plurality of containers with successive actuations of the assembly (e.g., pressing button 82 a number of times equal to the number of containers from which fluid is pumped) or with one actuation of the assembly (e.g., pressing button 82 once).

### EXAMPLES

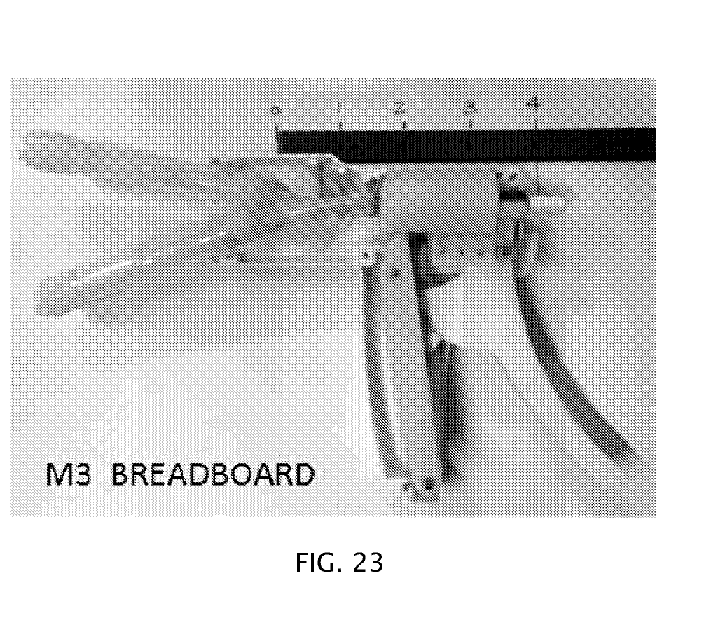

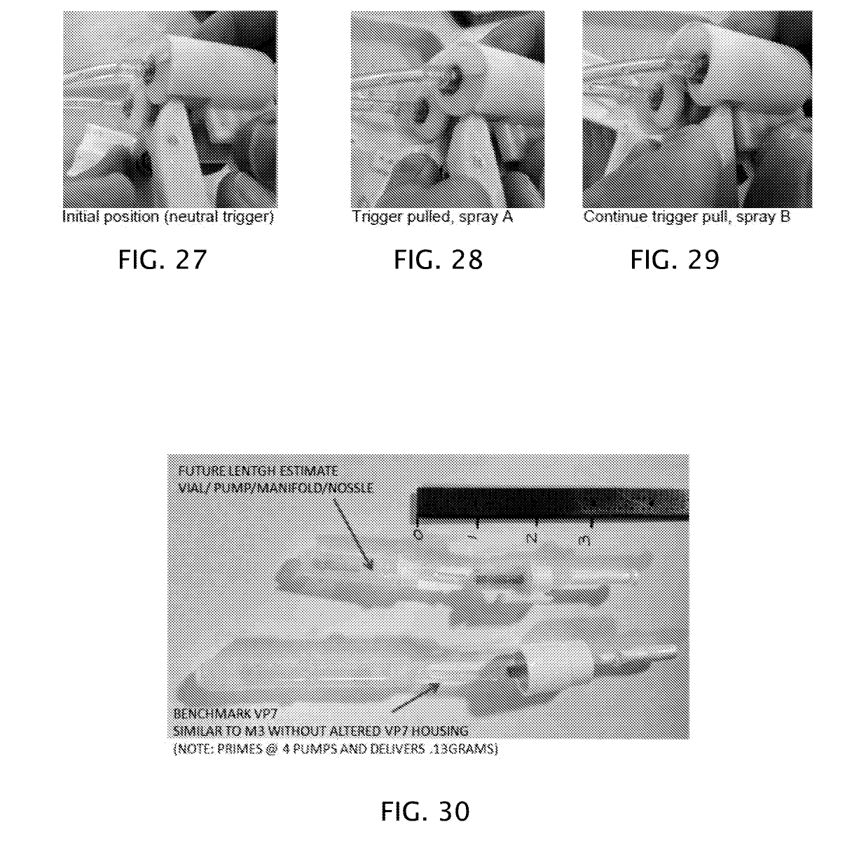

Described below is one example of experimental testing of a fluid delivery assembly prototype of the present disclosure. The prototype, components of the prototype, and other devices used in the experimental testing are depicted in FIGS. 23-30. The below example should not be interpreted to limit the scope of the claims or those embodiments described above; it is merely one implementation of the disclosed fluid delivery assemblies. The objective of the testing was to characterize the performance of the fluid delivery assembly prototype.

Metrics to be collected in the testing included:
Trigger actuation force;
Spray area and spray overlap;
Number of actuations to prime the fluid path;
Weight of fluid expelled during actuation;
Quality of spray; and
Duration of total spray.

Assumptions included:
Designing a device that included:
   Actuation of two pumps via a one-handed, single trigger pull;
   Re-arming of the device upon release of the trigger; and
   Sequential spraying from a container A, then from a container B;
Designing the device to target a surface at a distance of approximately 4 inches (10 centimeters);
Designing a spray target size of a 12 centimeters squared circle;
Designing the device to be manually actuated; and
Using the same priming technique for each test.

### Description of Prototype:

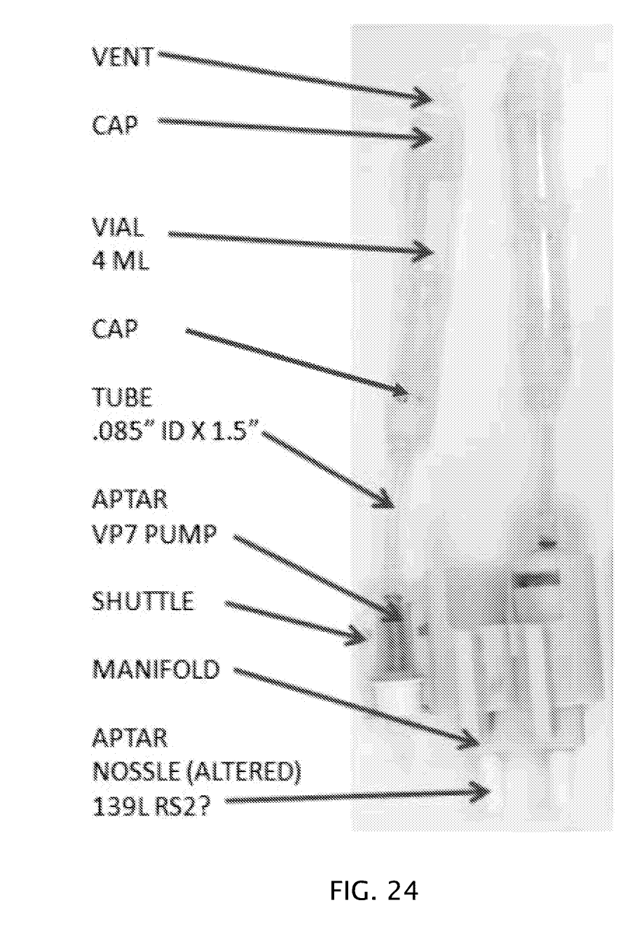

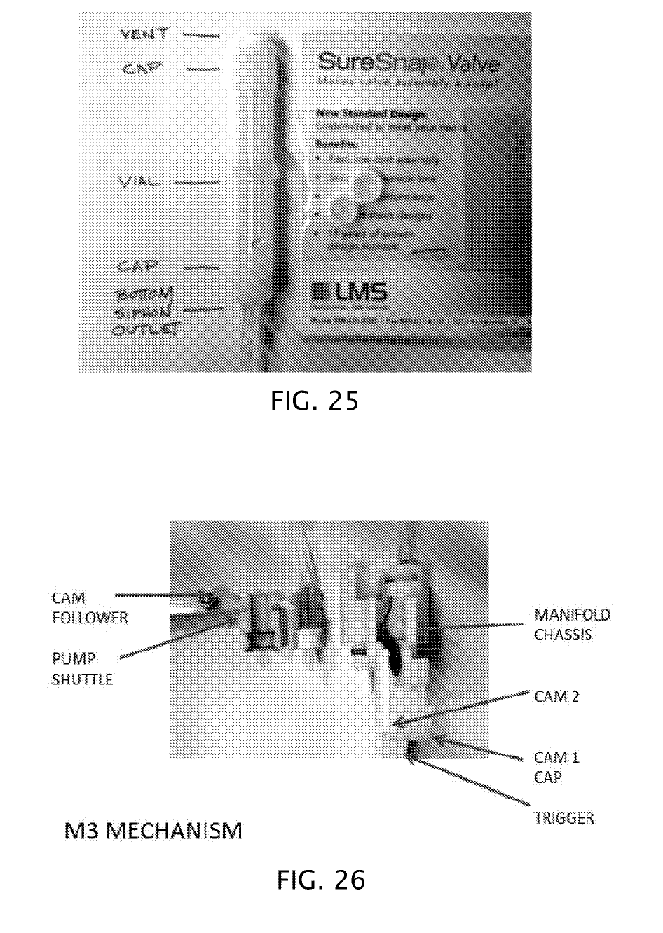

The prototype comprised a radial trigger staggered cam style spray mechanism tuned for sequential spray. The prototype utilized the Aptar VP7 pump and nozzle. The pump and nozzle were integrated into a custom housing with a custom fluid pathway. As depicted in FIG. 23, the nozzle fluid path ID was approximately 4 inches. As shown in FIG. 24, the pump was removed from the bottle cap such that air no longer vented into the fluid vessel. Upon installation of the pump, the vent path within the pump engine body and cap was aligned to encourage appropriate pump dosage. As in FIG. 25, the vial was configured to no longer utilize a top siphon tube, but it drew fluid directly out the non-vented bottom cap during use. The alternate cap vent was at the top and utilized a SureSnap LMS valve. As shown in FIG. 30, various fluid path lengths and venting can be used with the intent of using a standard OTS vial and cap. A benchmark Aptar VP7 device was made similarly to the prototype with a pump intact and a shortened nozzle length as shown in FIG. 30. This prototype demonstrates that this pump functions with an alternate vent scheme (e.g., a vial vent). The pump in this configuration was able to be primed in 4 pumps and delivered a 0.13 gram dose after priming. Comparison of the benchmark Aptar VP7 to the prototype demonstrated that the prototype could be further optimized. Variation in performance may not have been due to the venting or fluid path function.

### Test Methodology and Results:

Six tests were completed to evaluate the prototype. Modifications were made to the prototype between runs to address any issues that arose during testing. A summary of the methods and results is as follows:
Trigger actuation force: Trigger actuation force was measured by manually actuating the prototype and the benchmark Aptar VP7 with a force gauge. The force gauge was held stationary, and the devices were pressed into the force gauge. Actuation speed was intentionally varied from “slow” to “fast” to detect any change in peak actuation force due to actuation speed. Overall average trigger activation force was 2.435 kilograms. Overall finger actuation force of the benchmark Aptar VP7 with vented vials was 3.847 kilograms.
Spray Area and Spray Overlap: Spray area and overlap were observed by spraying two different colored water solutions onto paper. Standard printing paper was used to show target size. The device was sprayed perpendicular to the surface, 30° tilted right from the surface, and 30° degrees tilted back from the surface. Overlap of both fluids is feasible by targeting the nozzles such that their center line of spray action crosses at 4 inches (10 centimeters) from the nozzle exit. Spray patterns were generally circular and were similar to those of the benchmark Aptar VP7.
Number of Actuations to Prime Fluid Path: The number of actuations were counted until the spray pattern appeared full on the spray sheet. The device was measured after each actuation to determine if full prime had been achieved.
Weight of Fluid Expelled During Actuation: Dose volume was calculated by measuring the device weight (or change in device weight) between sprays. The prototype was capable of delivering 0.09-0.13 grams of fluid from the B (blue) side of the device. It was unable to be determined with accuracy the amount of fluid delivered from the A (red) side. The benchmark Aptar VP7 recorded a change in weight between 0.11 and 0.13 grams. Further, the impact of utilizing the device within a 30 degree cone was evaluated (tilting the device to the right/left, or 30 degrees back toward the user). Initial results show no obvious impact to the spray pattern or amount of fluid expelled. The fluid path and pump had access to both fluids in all positions of use.
Quality of Spray: Fine mist observed consistently except in cases of potential blockage.
Duration of Total Spray: More observations are required to determine duration of each spray independently.

It is anticipated that with optimization of materials and techniques that the prototype can be improved.

### Other Potential Prototypes and Considerations:

It is anticipated that, among other potential prototypes, the following characteristics of a fluid delivery assembly could be advantageous: a fixed nozzle, a fixed manifold, a pump shuttle, a pump linear movement when driven by cam, and flexible tubing behind pump (which can allow for pump action to support spray). Flexible tubing could attach to the vial connection (assuming a piercing needle). An automatic spring-loaded trigger return could also be integrated.

Another potential prototype could include: a fixed chassis with a pump, linear movement in shuttle nozzle (which can allow for spray actuation). The nozzle distance to the wound could change during spray (e.g., by 0.300 inches), impacting distance tolerance.

Prototypes could also:
Include water resistant materials (e.g., Delrin);
Avoid wicking adhesives for fittings to ensure proper plumbing (e.g., high-precision press fit);
Adapt the trigger and cam mechanism to allow for over-travel (e.g., for dispensed volume tuning and for feel during actuation);
Allow for tuning of the cam profile for dispense rate, pressure, and timing of spray;
Include a pump (or pumps) with a higher volume output (e.g., greater than 0.130 milliliters);
Allow for tuning of the initial cam engagement for Vial A;
Allow for tuning of the stagger for timing of cam engagement of Vial A, then Vial B;
Allow for selection of the spring that is engaged through full swing of pivot trigger and tuning overall travel of pivot trigger;
Allow for tuning of the trigger actuation to minimize the user's perception/feel of separate pumps;
Allowing some travel in the actuation prior to engagement with the pump(s) to allow the user's momentum to carry through the entire actuation stroke;
Include non-kinking tubing, or other tubing materials, between vial connection and pump;
Allow for minimization of return force applied by the tubing on the pump; and
Include rigid connection between vial and pump shuttle and allow vials to move linearly with pump action.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present assemblies and methods are not intended to be limited to the particular forms disclosed. Rather, they include all modifications and alternatives falling within the scope of the claims, and embodiments other than the one shown may include some or all of the features of the depicted embodiment. For example, components may be combined as a unitary structure and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A fluid delivery assembly comprising:
a first inlet channel;
a second inlet channel;
a first outlet channel;
a second outlet channel;
a first pump;
a second pump; and
a body configured to be coupled to a first container and a second container such that an interior of the first container is in fluid communication with the first inlet channel, and such that an interior of the second container is in fluid communication with the second inlet channel;
where if a first container with a fluid and a second container with a fluid, are coupled to the body, the assembly is configured upon at least one actuation to successively pump:
fluid from the first container, through the first inlet channel, and out the first outlet channel; and
fluid from the second container, through the second inlet channel, and out the second outlet channel.

2. The assembly of claim 1, where the fluid from the first container and the fluid from the second container is pumped with successive actuations of the assembly.

3. The assembly of claim 1, where the fluid from the first container and the fluid from the second container is pumped with one actuation of the assembly.

4. The assembly of claim 1, where the pump is configured to be actuated by pressing a button.

5. The assembly of claim 1, where the first and second outlet channels are adjacent.

6. The assembly of claim 1, where the first and second outlet channels are parallel.

7. The assembly of claim 1, where the first and second outlet channels extend from the body.

8. The assembly of claim 1, where at least one of the first and second outlet channels is configured to atomize a fluid.

9. The assembly of claim 1, where when the body is coupled to a first container and a second container, at least one of the first and second containers contains a solution.

10. The assembly of claim 9, where the solution comprises a suspension of cells.

11. The assembly of claim 9, where the solution comprises fibrinogen.

12. The assembly of claim 9, where the solution comprises thrombin.

13. The assembly of claim 9, where the solution is to be delivered to a body of a person.

14. The assembly of claim 4, further comprising:
a linear cam drive configured, upon pressing the button, to successively actuate the first pump and the second pump.

15. The assembly of claim 4, further comprising:
a rack drive configured, upon pressing the button, to successively actuate the first pump and the second pump.

16. The assembly of claim 4, further comprising:
a spring drive configured, upon pressing the button, to successively actuate the first pump and the second pump.

17. The assembly of claim 4, further comprising:
a rotary cam drive configured, upon pressing the button, to successively actuate the first pump and the second pump.

18. A fluid delivery assembly comprising:
a plurality of inlet channels;
a plurality of outlet channels;
a plurality of pumps; and
a body configured to be coupled to a plurality of containers such that an interior of the plurality of containers is in fluid communication with an inlet channel of the plurality of inlet channels;
where if a plurality of containers each having a fluid, are coupled to the body, the assembly is configured upon at least one actuation to successively pump fluid from each of the plurality of containers, through an inlet channel of the plurality of inlet channels, and out an outlet channel of the plurality of outlet channels.

19. The assembly of claim 18, where the fluid is pumped from each of the plurality of containers with successive actuations of the assembly.

20. The assembly of claim 18, where the fluid is pumped from each of the plurality of containers with one actuation of the assembly.

21. The assembly of claim 18, where the assembly is configured to be actuated by pressing a button.

22. The assembly of claim 18, where the plurality of outlet channels are adjacent.

23. The assembly of claim 18, where the plurality of outlet channels are parallel.

24. The assembly of claim 18, where the plurality of outlet channels extend from the body.

25. The assembly of claim 18, where at least one of the plurality of outlet channels is configured to atomize a fluid.

26. The assembly of claim 18, where when the body is coupled to a plurality of containers, at least one of the plurality of containers contains a solution.

27. The assembly of claim 26, where the solution comprises a suspension of cells.

28. The assembly of claim 26, where the solution comprises fibrinogen.

29. The assembly of claim 26, where the solution comprises thrombin.

30. The assembly of claim 26, where the solution is to be delivered to a body of a person.

31. The assembly of claim 21, further comprising:
a linear cam drive configured, upon pressing the button, to successively actuate the plurality of pumps.

32. The assembly of claim 21, further comprising:
a rack drive configured, upon pressing the button, to successively actuate the plurality of pumps.

33. The assembly of claim 21, further comprising:
a spring drive configured, upon pressing the button, to successively actuate the plurality of pumps.

34. The assembly of claim 21, further comprising:
a rotary cam drive configured, upon pressing the button, to successively actuate the plurality of pumps.

35. A fluid delivery assembly comprising:
a first inlet channel;
a second inlet channel;
a first outlet channel;
a second outlet channel;
at least one valve coupled to the first inlet channel, the second inlet channel, the first outlet channel, and the second outlet channel, the at least one valve configured to be actuated between:
a first configuration in which fluid communication is permitted between the first inlet channel and the first outlet channel and prevented between the second inlet channel and the second outlet channel; and
a second configuration in which fluid communication is permitted between the second inlet channel and the second outlet channel and prevented between the first inlet channel and the first outlet channel;
a body coupled to the at least one valve and configured to be coupled to a first container and a second container such that an interior of the first container is in fluid communication with the first inlet channel, and such that an interior of the second container is in fluid communication with the second inlet channel; and
at least one pump coupled to the body such that if a first container with a first fluid, and a second container with a second fluid, are coupled to the body, the at least one pump is configured to:
pump the first fluid from the first container, through the first inlet channel and the at least one valve, and out the first outlet channel if the at least one valve is in the first configuration; and
pump the second fluid from the second container, through the second inlet channel, and out the second outlet channel if the at least one valve is in the second configuration.

36. The assembly of claim 35, where the at least one valve alternates between the first and second configurations with successive actuations of the assembly.

37. The assembly of claim 35, where the at least one valve alternates between the first and second configurations with one actuation of the assembly.

38. The assembly of claim 35, where the assembly is configured to be actuated by pressing a button.

39. The assembly of claim 35, where the first and second outlet channels are adjacent.

40. The assembly of claim 35, where the first and second outlet channels are parallel.

41. The assembly of claim 35, where the first and second outlet channels extend from the body.

42. The assembly of claim 35, where at least one of the first and second outlet channels is configured to atomize a fluid.

43. The assembly of claim 35, where when the body is coupled to a first container and a second container, at least one of the first and second containers contains a solution.

44. The assembly of claim 43, where the solution comprises suspension of cells.

45. The assembly of claim 43, where the solution comprises fibrinogen.

46. The assembly of claim 43, where the solution comprises thrombin.

47. The assembly of claim 43, where the solution is to be delivered to a body of a person.

48. A fluid delivery assembly comprising:
a plurality of inlet channels;
a plurality of outlet channels;
at least one valve coupled to the plurality of inlet channels and the plurality of outlet channels, the at least one valve configured to be actuated between a plurality of configurations, each configuration permitting fluid communication between one of the plurality of inlet channels and one of the plurality of outlet channels and preventing fluid communication between the other(s) of the plurality of inlet channels and the other(s) of the plurality of outlet channels;
a body coupled to the at least one valve and configured to be coupled to a plurality of containers such that an interior of each of the plurality of containers is in fluid communication with an inlet channel of the plurality of inlet channels; and
at least one pump coupled to the body such that if a plurality of containers with fluid are coupled to the body, the at least one pump is configured to pump from each of the plurality of containers, through an inlet channel of the plurality of inlet channels, and out of an outlet channel of the plurality of outlet channels in each of the plurality of configurations.

49. The assembly of claim 48, where the at least one valve alternates between the plurality of configurations with successive actuations of the assembly.

50. The assembly of claim 48, where the at least one valve alternates between the plurality of configurations with one actuation of the assembly.

51. The assembly of claim 48, where the assembly is configured to be actuated by pressing a button.

52. The assembly of claim 48, where the plurality of outlet channels are adjacent.

53. The assembly of claim 48, where the plurality of outlet channels are parallel.

54. The assembly of claim 48, where the plurality outlet channels extend from the body.

55. The assembly of claim 48, where at least one of plurality of outlet channels is configured to atomize a fluid.

56. The assembly of claim 48, where when the body is coupled to a plurality of containers containing fluid, at least one of the plurality of containers contains a solution.

57. The assembly of claim 56, where the solution comprises a suspension of cells.

58. The assembly of claim 56, where the solution comprises fibrinogen.

59. The assembly of claim 56, where the solution comprises thrombin.

60. The assembly of claim 56, where the solution is to be delivered to a body of a person.

61. A method of delivering fluid to a person's body comprising:
coupling a plurality of containers containing fluid to a fluid delivery assembly, where the fluid delivery assembly comprises:
a plurality of inlet channels;
a plurality of outlet channels;
at least one valve, where the at least one valve is configured to alternate between a plurality of configurations, each configuration permitting fluid communication between one of the plurality of inlet channels and one of the plurality of outlet channels and preventing fluid communication between the other(s) of the plurality of inlet channels and the other(s) of the plurality of outlet channels; and
at least one pump; and
actuating the at least one pump to successively pump from each of the plurality of containers, through a corresponding inlet channel, and out of an outlet channel.

62. The method of claim 61, where the at least one valve alternates between the plurality of configurations with successive actuations of the assembly.

63. The method of claim 61, where the at least one valve alternates between the plurality of configurations with one actuation of the assembly.

64. The method of claim 61, where the assembly is configured to be actuated by pressing a button.

65. The method of claim 61, where at least one of the plurality of containers contains a solution.

66. The method of claim 65, where the solution comprises suspension of cells.

67. The method of claim 65, where the solution comprise fibrinogen.

68. The method of claim 65, where the solution comprises thrombin.

69. The method of claim 65, where the solution is to be delivered to a body of a person.

70. A method of delivering fluid to a person's body comprising:
coupling a plurality of containers containing fluid to a fluid delivery assembly, where the fluid delivery assembly comprises:
a plurality of inlet channels;
a plurality of outlet channels; and
a plurality of pumps; and
actuating the plurality of pumps to successively pump from each of the plurality of containers, through a corresponding inlet channel, and out of an outlet channel.

71. The method of claim 70, where the plurality of pumps pump from each of the plurality of containers with successive actuations of the assembly.

72. The method of claim 70, where the plurality of pumps pump from each of the plurality of containers with one actuation of the assembly.

73. The method of claim 70, where the assembly is configured to be actuated by pressing a button.

74. The method of claim 70, where at least one of the plurality of containers contains a solution.

75. The method of claim 74, where the solution comprises suspension of cells.

76. The method of claim 74, where the solution comprise fibrinogen.

77. The method of claim 74, where the solution comprises thrombin.

78. The method of claim 74, where the solution is to be delivered to a body of a person.
